(19) United States
(12) Patent Application Publication
(10) Pub. No.: US 2005/0202025 A1
(43) Pub. Date: Sep. 15, 2005

(54) Antibodies to the FbsA protein of streptococcus agalactiae and their use in treating or preventing infections

(76) Inventors: Pietro Speziale, Pavia (IT); Giampiero Pietrocola, Pavia (IT); Livia Visai, Rosate (Milano) (IT)

Correspondence Address: STITES & HARBISON PLLC, 1199 NORTH FAIRFAX STREET, SUITE 900, ALEXANDRIA, VA, 22314, US

(21) Appl. No.: 10/897,429

(22) Filed: Jul. 23, 2004

Related U.S. Application Data
(60) Provisional application No. 60/489,098, filed on Jul. 23, 2003.

(51) Int. Cl. G01N 33/554; G01N 33/569; A61K 39/40

(52) U.S. Cl. 424/165.1; 530/388.4; 435/7.32

(57) ABSTRACT

Monoclonal and polyclonal antibodies are provided which can bind to the FbsA protein of Streptococcus agalactiae (GBS) and which can be used to prevent adherence of the bacteria to host cells and thus be useful in the treatment and protection against infection from S. agalactiae. The antibodies of the invention can also be raised against the fibrinogen binding domain of FbsA or the repeat region therein, and in addition to preventing bacterial adherence, the antibodies to FbsA are advantageous in that they can be used to prevent platelet aggregation and thrombus formation.

### CROSS REFERENCE TO RELATED APPLICATIONS

The present application claims the benefit of U.S. provisional application Ser. No. 60/489,098 filed Jul. 23, 2003, incorporated herein by reference.

### FIELD OF THE INVENTION

The present invention relates in general to antibodies that can recognize and bind to the fibrinogen-receptor protein FbsA from Streptococcus agalactiae (also known as Group B streptococci or GBS), and in particular to antibodies which are preferably generated against the fibrinogen binding domain of FbsA and its repeat region and which can be used to prevent adherence of S. agalactiae to host cells so as to protect against infection. In addition, the invention also relates to the use of FbsA antibodies in inhibiting bacteria-induced platelet aggregation so as to assist in combating thrombus formation caused by streptococcal infection.

### BACKGROUND OF THE INVENTION

Streptococcus agalactiae (also known as group B streptococcus or GBS) is a bacteria pathogen which is a major cause of a number of serious and possibly life-threatening diseases. Further, S. agalactiae is a frequent colonizer of the gastrointestinal and urogenital tract of humans (3), and is also the cause of substantial pregnancy-related morbidity and has emerged as an increasingly common cause of invasive disease in the elderly and in immunocompromised persons (55). In addition, S. agalactiae is the most common cause of bacterial pneumonia, sepsis and meningitis in human newborns (3). Neonates acquire S. agalactiae from colonized mothers by aspiration of infected amniotic fluid or vaginal secretions at birth, followed by bacterial adherence to pulmonary epithelial cells (47).

Previous studies have confirmed that the adherence of the bacteria to lung epithelial cells is a prerequisite for the invasion of deeper tissues and the dissemination of the bacteria to the bloodstream, and several studies have demonstrated the adherence of S. agalactiae to epithelial cells both in vitro and in vivo (5, 42, 49, 61). However, the underlying mechanisms of this interaction are only poorly understood, thus making it very difficult at present to develop successful methods of preventing bacterial adherence and infection. For example, lipoteichoic acid (LTA) was initially postulated to mediate the adherence of S. agalactiae to epithelial cells (32, 33), but later studies demonstrated a cytotoxic effect of LTA on eukaryotic cells (15). While certain pretreatments, such as protease, can decrease the bacterial adherence to host cells of S. agalactiae (31, 49), surface proteins are presently assumed to be important for this process. However, because the bacterial determinants that promote adherence of S. agalactiae to epithelial cells have not been elucidated, it has been difficult to focus on the crucial elements of the adherence and even more difficult to overcome them.

It has also been known that numerous pathogenic bacteria adhere to host cells by surface proteins, termed adhesins or MSCRAMM®s, that bind to components of the extracellular matrix (ECM). The ECM of mammalian tissues consists of glycoproteins, including collagen, laminin, fibronectin and fibrinogen, which form a macromolecular structure underlying epithelial and endothelial cells (21). Accordingly, ECM's have often been a subject of interest with regard to GBS, and several studies have described interactions of S. agalactiae with ECM proteins such as laminin, fibronectin, and fibrinogen (26, 48, 51). In addition, two fibrinogen-binding proteins from S. agalactiae, termed FbsA and FbsB, respectively, have been identified (19, 44). However, as with many of the ECM proteins, it has still remained a problem to identify and utilize the information concerning the exact nature of the mechanisms behind bacterial adherence to these proteins and the resulting infection. It thus it has been very difficult to accurately assess in every case the binding of bacteria to the different ECM proteins because it varies in every case, and in most cases, the underlying mechanisms are only poorly understood.

It is thus a highly desirable object to obtain detailed information with regard to the adherence between bacteria and the different ECM proteins, such as FbsA, and to utilize this information to develop antibodies and methods that can be effective in blocking adherence of GBS to human and animal host cells and in treating and preventing GBS infections.

Another problem that arises in conjunction with certain S. agalactiae infections is that these infections can trigger platelet aggregation and thrombus formation, such as in streptococcal endocarditis. Once again, the exact mechanisms and causes of the platelet aggregation resulting from GBS infection has not been well known and thus it has continued to be difficult to develop effective therapeutic regimens for treating or preventing such aggregation.

It thus remains a highly desirable object to obtain a better understanding of the mechanisms behind the ability of GBS to cause platelet aggregation and thrombus formation under certain disease conditions, and to develop compositions and methods which will be effective in inhibiting platelet aggregation.

### SUMMARY OF THE INVENTION

Accordingly, it is an object of the present invention to provide antibodies that can bind to the FbsA protein from S. agalactiae so as to prevent bacterial adherence to host cells.

It is also an object of the present invention to provide isolated monoclonal and polyclonal antibodies which can recognize the FbsA protein and which are useful in methods to treat, prevent or diagnose GBS infections.

It is another object of the present invention to provide antibodies which can bind specifically to the fibrinogen-binding domain of the FbsA protein of S. agalactiae and which are also useful in preventing or inhibiting bacterial adherence to host cells and thus can be used to treat or prevent GBS infections.

It is yet another object of the present invention to provide monoclonal antibodies to the fibrinogen binding domain of the FbsA protein which can be useful in preventing adherence of Streptococcal bacteria by inhibiting or impairing the binding of the FbsA protein to fibrinogen.

It is further an object of the present invention to provide antibodies and antisera which can recognize the fibrinogen binding domain of the FbsA protein, and which can thus be useful in methods of treating, preventing, identifying or diagnosing streptococcal infections.

It is still further an object of the invention to provide antibodies and compositions which can be useful in inhibiting platelet aggregation and the resulting thrombus formation that accompanies certain GBS disease conditions such as endocarditis.

These and other objects are provided by virtue of the present invention which comprises the generation and use of isolated monoclonal and polyclonal antibodies which can recognize the S. agalactiae FbsA fibrinogen-binding protein and/or its fibrinogen-binding domains, for the blocking of the adherence of S. agalactiae and the treatment or prevention of Streptococcus infections. The present application also comprises the generation of monoclonal antibodies against the fibrinogen-binding domain of the FbsA protein of GBS which are effective in blocking adherence and thus treating and preventing GBS infection, as well as therapeutic compositions and antisera containing such antibodies. Still further, the present invention provides a means of using these antibodies in the prevention and treatment of platelet aggregation and can thus be useful in pathogenic conditions such as endocarditis wherein it is necessary to inhibit or reverse thrombus formation.

These embodiments and other alternatives and modifications within the spirit and scope of the disclosed invention will become readily apparent to those skilled in the art from reading the present specification and/or the references cited herein, all of which are incorporated by reference.

### BRIEF DESCRIPTION OF THE DRAWING FIGURES

The descriptions of the drawing figures are included below as follows: FIG. 1. Time course of S. agalactiae O90R adherence to human fibrinogen-coated microtiter wells, wherein cells of S. agalactiae O90R (5×10⁷) were incubated with immobilized fibrinogen (10 micrograms/ml) for the periods of time indicated. After washing with PBST (PBS containing 0.05% Tween 20) the wells were incubated with 0.5 micrograms of rabbit anti-whole S. agalactiae cells IgG for 90 min at 22° C. Unbound bacteria were removed by washing the wells five times with PBST. Antibody bound to bacteria was detected by incubation of the wells for 1 h with 1:1000 dilution of peroxidase-conjugated goat anti-rabbit IgG. After washing the conjugated enzyme was reacted with o-phenylenediamine dihydrochloride and the absorbance at 490 nm was monitored with a microplate reader.

FIG. 2. Expression of fibrinogen-binding activity by S. agalactiae O90R. Bacterial cells were grown for increasing periods of time, harvested and assayed for adherence to immobilized fibrinogen. Bacterial attachment was detected incubating the wells with 0.5 microgram of immune IgG against whole cells of S. agalactiae.

FIG. 3. Saturability of fibrinogen binding to Streptococcus agalactiae. Overnight-grown S. agalactiae O90R cells were washed and resuspended in 50 mM carbonate buffer, pH 9.6, and then 5×10⁷ cells in 100 microliters were used to coat microtiter plate wells (in triplicate). Bacteria were allowed to bind at 37° C overnight. Wells were washed five times with PBST to remove the non-adherent cells, and the remaining areas of the wells were blocked with 2% BSA in PBST. Increasing amounts of fibrinogen (panel A) or fragment D (panel B) were added to each well in 100 microliters of 1% BSA in PBS and incubated for 90 min at 22° C. The wells were washed five times with PBST and incubated with 0.5 micrograms of human fibrinogen-specific mouse antibody for 90 min. After extensive washing, the wells were added of 100 microliters of a 1:1000 dilution of rabbit anti-mouse IgG conjugated to horseradish peroxidase. After washing, the conjugated enzyme was reacted with o-phenylenediamine dyhydrochloride and the absorbance at 490 nm was monitored with a microplate reader.

FIG. 4. Localization of fibrinogen-binding site on fibrinogen. Bacteria (5×10⁷) (panel A) or FbsA-N (0.5 microgram /well) (panel B) were coated on microtiter wells and allowed to incubate with either intact fibrinogen, fragment D or fragment E (0.5 micrograms/well). After washing the wells to remove unbound ligand, bound fibrinogen or fragment was probed with 0.5 micrograms of a mouse anti-human fibrinogen IgG for 90 min. The amount of IgG bound was detected by addition of 1:1000 dilution of peroxidase-conjugated rabbit anti-mouse antibody.

FIG. 5. Amino acid sequence of pep-FbsA.

FIG. 6. Inhibitory activity of anti-pep-FbsA antibody on streptococcal adherence to fibrinogen. Microtiter wells were coated with human fibrinogen (1 microgram in 100 microliters). S. agalactiae cells (5×10⁷ cells /well) were preincubated with increasing amounts of indicated IgG before being added to the wells. Adherent bacteria were probed with 0.5 micrograms of a rabbit IgG against whole cells of S. agalactiae. After washing, antibody bound to bacteria was detected by addition to the wells of a goat anti-rabbit peroxidase-conjugated polyclonal antibody and subsequent addition of a chromogenic substrate.

FIG. 7. MAb specificity for repeat unit of FbsA. Microtiter wells were coated with the indicated proteins (1 microgram/well) and then probed with 1 microgram of each mAb. To detect binding of the antibody the plates were washed and incubated with 1:1000 dilution of a rabbit anti-mouse peroxidase-conjugated antibody.

FIG. 8. Effect of mAbs anti FbsA on the binding of fibrinogen to FbsA. Recombinant FbsA-N was immobilized onto microtiter wells (0.5 micrograms in 100 microliters) and probed with biotin-labelled fibrinogen in the presence of 7.5 micrograms of each mAb. After washing with PBST, binding of the ligand was quantitated by adding 1:2000 dilution of avidin-conjugated peroxidase and developed with o-phenylenediamine hydrochloride.

FIG. 9. Concentration-dependent effect on biotin-labelled fibrinogen binding to immobilized FbsA by mAbs 5H2 and 2B1. Microtiter wells were coated with FbsA-N (0.5 micrograms in 100 microliters) and incubated with biotin-labelled fibrinogen in the presence of increasing amounts of indicated mAbs. Fibrinogen binding was quantitated as described in FIG. 8.

FIG. 10. Effect of mAbs on the attachment of S. agalactiae O90R to fibrinogen. Streptococcal cells (5×10⁷) were preincubated with 7.5 micrograms of mAbs 5H2 and 2B1 and then added to microtiter plates coated with fibrinogen (1 microgram/well). After washing adherent bacteria were detected as reported in FIG. 6.

FIG. 11. Dose-dependent effects on the attachment of S. agalactiae O90R to fibrinogen. S. agalactiae cells (5×10⁷) were preincubated with increasing concentrations of mAbs 5H2 and 2B1 before being added to fibrinogen coated wells. Adherent cells were detected as reported in FIG. 6.

FIG. 12. Platelet aggregation induced by S. agalactiae strains. The ability of S. agalactiae and their corresponding mutants (delta FbsA) to activate platelet aggregation in PRP was tested. The results are presented as percentage aggregation.

FIG. 13. Inhibition of ADP-induced platelet aggregation by FbsA-N. To investigate the ability of FbsA-N to interfere with platelet aggregation platelet rich plasma (PRP) (0.4 ml) was preincubated for 5 min with increasing concentrations of FbsA-N and then stimulated with ADP (10 micromoles /L). The aggregation traces are from one experiment, representative of 3 total.

FIG. 14. Effects of inhibitors on S. agalactiae 6313- induced platelet aggregation. Inhibition of S. agalactiae 6313- induced platelet aggregation by GPIIb /IIIa. Platelet rich plasma (0.4 ml) was pretreated with RGDS (1 millimole/L), PGE₁ (1 micromole/L) or apyrase (10 U/ml) for 10 min or with ASA (1 micromole/L) for 30 min before the addition of the agonist (ADP, 10 micromoles/L) (blue columns) or S. agalactiae 6313 cells (5×10⁷) (white columns). Results are expressed as percentage of aggregation.

FIG. 15. Effect of total plasma and fibrinogen on gel filtered platelet aggregation.

FIG. 16. Inhibitory effect of mAb 5H2 on platelet aggregation induced by S. agalactiae 6313. 50 microliters of S. agalactiae cells (5×107) (grown to stationary phase), were preincubated with 5 micrograms of 5H2 or an equal concentration of isotype-matched control mAb (2B), and then tested for their ability to activate aggregation in PRP (0.4 ml).

FIG. 17. Neutralizing effect of mAb 5H2 on the inhibition of platelet aggregation by FbsA-N. Platelets from one donor were incubated for 5 min with FbsA-N (0.64 micromoles/L) in the presence of increasing amounts of anti pep-FbsA mAb 5H2 and then stimulated with ADP (10 micromoles/L). Data are representative of 3 experiments.

FIG. 18. Binding of radiolabelled fibrinogen (A), and host cell adherence and invasion (B) by different S. agalactiae strains and their fbsA deletion mutants. Binding of ¹²⁵I-labelled fibrinogen was quantitated by incubating a defined number of bacteria with a defined amount of radiolabelled fibrinogen, and relating the amount of bacteria-bound fibrinogen to the total amount of fibrinogen added. To determine the adherence and invasiveness of the different strains with the lung epithelial cell line A549, equal numbers of each streptococcal strain were used to infect A549 cells, and the number of cell adherent and internalized bacteria was related to the number of input bacteria. Each experiment was performed at least three times in triplicate.

FIG. 19. Adherence and invasion of the lung epithelial cell line A549 by the S. agalactiae strains 6313 pOri23, 6313 DfbsA pOri23, and 6313 DfbsA pOrifbsA, and by the lactococcal strains L. lactis pOri23 and L. lactis pOrifbsA, respectively. The epithelial cell line A549 was infected with an equal amount of bacteria of each strain, and the number of cell adherent and internalized bacteria was related to the number of input bacteria. The dotted line separates the results obtained with S. agalactiae and L. lactis from each other. Each experiment was performed at least three times in triplicate.

FIG. 20. Detection of FbsA-binding to the surface of A549 cells by flow cytometry. A549 cells were incubated with different amounts of purified FbsA fusion protein and tested with anti-his-tag antibodies and anti-mouse-FITC coupled antibodies for the interaction of FbsA with the host cell surface.

FIG. 21. Binding of FbsA-coated latex beads to human A549 cells. Latex beads were either coated with BSA (A) or FbsA fusion protein (B-D) and the interaction of the coated beads with the lung epithelial cell line A549 was analyzed by scanning electron microscopy

FIG. 22. Competitive inhibition of streptococcal adherence and invasion by the monoclonal antibody 5H2 (mAb 5H2), which specifically blocks the binding of FbsA to human fibrinogen. Tissue culture experiments were performed after pretreatment of S. agalactiae 6313 with different amounts of mAb 5H2. Each experiment was performed at least three times in triplicate.

### DETAILED DESCRIPTION OF THE PREFERRED EMBODIMENTS

In accordance with the present invention, there are provided isolated and/or purified antibodies which bind to the FbsA protein from S. agalactiae, and as set forth in more detail below, these antibodies may be polyclonal or monoclonal, and they may be used so as to prevent adherence of S. agalactiae to host cells, and more particularly to prevent adherence of S. agalactiae to fibrinogen. As set forth below, these antibodies may also be raised against, or generated so as to specifically bind with, active fragments of the FbsA protein, including the fibrinogen binding domain of FbsA, as well as to the repeat region therein, and these antibodies may be used in a number of ways including the treatment and prevention of GBS infection as well as methods of preventing platelet aggregation in a patient in need of such therapy.

In one aspect of the present invention, isolated and/or purified monoclonal antibodies are provided which can bind to the FbsA protein from S. agalactiae and/or its fibrinogen binding regions, and such antibodies can be useful in methods of preventing adherence of S. agalactiae to host cells and thus treat or prevent a streptococcal infection when used in amounts effective to prevent or treat such infections. These monoclonal antibodies may be produced using conventional means, e.g., the method of Kohler and Milstein, Nature 256:495-497 (1975), or other suitable ways known in the field, and in addition can be prepared as chimeric, humanized, or human monoclonal antibodies in ways that would be well known in this field. Still further, monoclonal antibodies may be prepared from a single chain, such as the light or heavy chains, and in addition may be prepared from active fragments of an antibody which retain the binding characteristics (e.g., specificity and/or affinity) of the whole antibody. By active fragments is meant an antibody fragment which has the same binding specificity as a complete antibody which binds to a fibrinogen binding protein, and the term “antibody” as used herein is meant to include said fragments. Additionally, antisera prepared using monoclonal or polyclonal antibodies in accordance with the invention are also contemplated and may be prepared in a number of suitable ways as would be recognized by one skilled in the art.

As indicated above, antibodies in accordance with the invention may be prepared in a number of suitable ways that would be well known in the art, such as the well-established Kohler and Milstein method described above which can be utilized to generate monoclonal antibodies. In addition, as set forth above, the antibodies may be generated to bind with the FbsA protein or active fragments thereof including the fibrinogen binding domain and/or its repeat region. In one exemplary method of generating monoclonal antibodies in accordance with the invention, a peptide comprising the repeat region of FbsA (“Pep-FbsA”) was coupled to KLH so as to produce the desired mAbs. In this procedure, BALB/c mice and the mouse myeloma line Spe/0 Ag.14 were used. Hybridoma were screened 10 days postfusion by ELISA for recognition of pep-FbsA coupled to ovalbumin (pep-OVA), and positive hybridomas were rescreened for recognition of the synthetic repeat unit of FbsA. A number of hybridomas that gave a strong ELISA response to pep-OVA were cloned by limiting dilution and the hybridomas 2B1, 5C9, 5H2 and 10H1 were selected and grown to high density in RPMI 1640 medium containing 10% (v/v) fetal bovine serum and antibiotics. The antibody isotype was determined by using the reagent provided with Bio-Rad mouse hybridoma Isotyper Kit. Mabs 5C9, 5H2 and 10H1 were IgG₁₋ₖ and mAb 2B1 was an IgG_{2b-k}. Accordingly, in accordance with the invention, monoclonal antibodies can thus be produced which bind to the FbsA protein of S. agalactiae and which can be used to block the adherence of S. agalactiae to fibrinogen.

Although production of antibodies as indicated above is preferably carried out using synthetic or recombinantly produced forms of the FbsA protein or its active peptide regions, antibodies may be generated from natural isolated and purified FbsA peptides or proteins. Still other conventional ways are available to generate the FbsA antibodies of the present invention using recombinant or natural purified FbsA proteins or their active regions, as would be recognized by one skilled in the art.

In addition to monoclonal antibodies, polyclonal antibodies that can bind to FbsA and thus be used to prevent adherence of FbsA to fibrinogen and host cells is another aspect of the invention. As one skilled in the art would recognize, there are a number of suitable ways of preparing polyclonal antibodies, and these methods generally involve injection of an immunogenic amount of FbsA and/or its active fragments (e.g., the repeat region as set forth above) into a suitable host animal, allowing sufficient time for the generation of polyclonal antibodies in the animal, and the isolation, collection and/or purification of the polyclonal antibodies from the host animal. In one such suitable procedure, mouse antisera containing polyclonal antibodies capable of recognizing FbsA was generated by immunization using the synthetic peptide of the repeat unit of FbsA (anti-pep-FbsA) which was coupled to KLH and intraperitoneally injected in BALB/c mice.

As would be recognized by one skilled in the art, the antibodies of the present invention may also be formed into suitable pharmaceutical compositions for administration to a human or animal patient in order to block adherence of S. agalactiae to host cells so as treat or prevent an S. agalactiae infection. Pharmaceutical compositions containing the antibodies of the present invention, or effective fragments thereof, may be formulated in combination with any suitable pharmaceutical vehicle, excipient or carrier that would commonly be used in this art, including such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art. Suitable methods of administration of any pharmaceutical composition disclosed in this application include, but are not limited to, topical, oral, anal, vaginal, intravenous, intraperitoneal, intramuscular, subcutaneous, intranasal and intradermal administration. In the desired composition, the composition will contain an effective amount of antibody so as to be useful in the methods as described further below.

For topical administration, the composition is formulated in the form of an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical formulations may also contain conventional carriers such as cream or ointment bases, ethanol, or oleyl alcohol.

Additional forms of antibody compositions, and other information concerning compositions, methods and applications with regard to other MSCRAMM®s will generally also be applicable to the present invention are disclosed, for example, in U.S. Pat. No. 6,288,214 (Hook et al.), incorporated herein by reference.

The antibodies and antibody compositions of the present invention may also be administered with a suitable adjuvant in. an amount effective to enhance the immunogenic response. For example, suitable adjuvants may include alum (aluminum phosphate or aluminum hydroxide), which is used widely in humans, and other adjuvants such as saponin and its purified component Quil A, Freund's complete adjuvant, RIBBI adjuvant, and other adjuvants used in research and veterinary applications. Still other chemically defined preparations such as muramyl dipeptide, monophosphoryl lipid A, phospholipid conjugates such as those described by Goodman-Snitkoff et al. J. Immunol. 147:410-415 (1991) and incorporated by reference herein, encapsulation of the conjugate within a proteoliposome as described by Miller et al., J. Exp. Med. 176:1739-1744 (1992) and incorporated by reference herein, and encapsulation of the protein in lipid vesicles such as Novasome™ lipid vesicles (Micro Vescular Systems, Inc., Nashua, N.H.) may also be useful.

In any event, the antibody compositions of the present invention will thus be useful for interfering with, modulating, or inhibiting binding interactions between S. agalactiae and fibrinogen on host cells and indwelling medical device and implants, and thus have particular applicability in developing compositions and methods of preventing or treating streptococcal infection.

Medical devices or polymeric biomaterials and implants that can be coated with the antibodies and compositions described herein include, but are not limited to, staples, sutures, replacement heart valves, cardiac assist devices, hard and soft contact lenses, intraocular lens implants (anterior chamber or posterior chamber), other implants such as corneal inlays, kerato-prostheses, vascular stents, epikeratophalia devices, glaucoma shunts, retinal staples, scleral buckles, dental prostheses, thyroplastic devices, laryngoplastic devices, vascular grafts, soft and hard tissue prostheses including, but not limited to, pumps, electrical devices including stimulators and recorders, auditory prostheses, pacemakers, artificial larynx, dental implants, mammary implants, penile implants, cranio/facial tendons, artificial joints, tendons, ligaments, menisci, and disks, artificial bones, artificial organs including artificial pancreas, artificial hearts, artificial limbs, and heart valves; stents, wires, guide wires, intravenous and central venous catheters, laser and balloon angioplasty devices, vascular and heart devices (tubes, catheters, balloons), ventricular assists, blood dialysis components, blood oxygenators, urethral/ureteral/urinary devices (Foley catheters, stents, tubes and balloons), airway catheters (endotracheal and tracheostomy tubes and cuffs), enteral feeding tubes (including nasogastric, intragastric and jejunal tubes), wound drainage tubes, tubes used to drain the body cavities such as the pleural, peritoneal, cranial, and pericardial cavities, blood bags, test tubes, blood collection tubes, vacutainers, syringes, needles, pipettes, pipette tips, and blood tubing.

It will be understood by those skilled in the art that the term “coated” or “coating”, as used herein, means to apply the antibody or active fragment, or pharmaceutical composition derived therefrom, to a surface of the device, preferably an outer surface that would be exposed to streptococcal bacterial infection. The surface of the device need not be entirely covered by the protein, antibody or active fragment.

In accordance with the present invention, immunogenic amounts of the FbsA protein and/or its active fragments as discussed above may be prepared as active vaccines for human and animal hosts in need of such vaccines.

As would be recognized by one skilled in this art, active vaccines in accordance with the present invention will employ an immunogenic amount of the FbsA protein or an active fragment thereof along with a suitable pharmaceutically acceptable vehicle, carrier or excipient. By immunogenic amount is meant a non-toxic amount of the protein or fragment which will elicit antibodies to FbsA in the host, and it is desired that a suitable amount of the immunogen be provided so as to obtain the desired therapeutic effect, e.g., treating or preventing a GBS infection. Accordingly, such amounts will vary in each case, and as one skilled in the art would recognize, the appropriate amount for any given vaccine will depend on a variety of conditions, including age, size and condition of the patient, and the nature of the bacterial infection being treated. As would also be recognized by one skilled in the art, vaccines in accordance with the invention may be packaged for administration in a number of suitable ways, such as by parenteral (i.e., intramuscular, intradermal or subcutaneous) administration or nasopharyngeal (i.e., intranasal) administration. One such mode is where the vaccine is injected intramuscularly, e.g., into the deltoid muscle, however, the particular mode of administration will depend on the nature of the bacterial infection to be dealt with and the condition of the patient. The vaccine is preferably combined with a pharmaceutically acceptable carrier to facilitate administration, and the carrier is usually water or a buffered saline, with or without a preservative. The vaccine may be lyophilized for resuspension at the time of administration or in solution.

The isolated antibodies of the present invention, or active fragments thereof, may also be utilized in the development of vaccines for passive immunization against GBS infections. Further, when administered as pharmaceutical composition to a wound or used to coat medical devices or polymeric biomaterials in vitro and in vivo, the antibodies of the present invention, may be useful in those cases where there is a previous GBS infection because of the ability of this antibody to further restrict and inhibit GBS binding to fibrinogen and thus limit or reduce the extent and spread of the infection. In addition, the antibody may be modified as necessary so that, in certain instances, it is less immunogenic in the patient to whom it is administered. For example, if the patient is a human, the antibody may be “humanized” by transplanting the complimentarity determining regions of the hybridoma-derived antibody into a human monoclonal antibody as described, e.g., by Jones et al., Nature 321:522-525 (1986) or Tempest et al. Biotechnology 9:266-273 (1991) or “veneered” by changing the surface exposed murine framework residues in the immunoglobulin variable regions to mimic a homologous human framework counterpart as described, e.g., by Padlan, Molecular Imm. 28:489-498 (1991), these references incorporated herein by reference. Even further, when so desired, the antibodies of the present invention may be administered in conjunction with a suitable antibiotic to further enhance the ability of the present compositions to fight bacterial infections.

In accordance with the present invention, methods are provided for preventing or treating a GBS infection which comprise administering an effective amount of the antibody of the present invention as described above in amounts effective to block adherence of GBS to host cells. In addition, these antibodies can be utilized in methods wherein the antibody is administered to a patient in need of such treatment in an amount effective to treat or prevent a GBS infection.

Accordingly, in accordance with the invention, administration of the antibodies of the present invention in any of the conventional ways described above (e.g., topical, parenteral, intramuscular, etc.), and will thus provide an extremely useful method of blocking adherence of S. agalactiae to fibrinogen and thus treating or preventing streptococcal infections in human or animal patients. In this context, by effective amount is meant that level of use, such as of an antibody titer, that will be sufficient to prevent adherence of the bacteria to fibrinogen, to inhibit binding of GBS to host cells and/or to be useful in the treatment or prevention of a GBS infection. As would be recognized by one of ordinary skill in this art, the level of antibody titer needed to be effective in treating or preventing such infections will vary depending on the nature and condition of the patient, and/or the severity of any the pre-existing infections.

In addition to the use of antibodies to the invention to prevent adherence of GBS to host cells and thus treat or prevent infection as described above, the present invention contemplates the use of these antibodies in additional ways, including the detection of GBS to diagnose an infection, whether in a patient or on medical equipment which may also become infected, and in methods of preventing or reducing platelet aggregation as will be described further below. In accordance with the invention, a preferred method of detecting the presence of GBS infections involves the steps of obtaining a sample suspected of being infected by GBS, such as a sample taken from an individual, for example, from one's blood, saliva, tissues, bone, muscle, cartilage, or skin, introducing the GBS antibodies as set forth above to the sample, and determining if there is any binding between the antibodies and the sample. Such diagnostic assays which can utilize the antibodies of the present invention are well known to those skilled in the art and include methods such as radioimmunoasssay, Western blot analysis and ELISA assays. In these and other assays, various labels may be placed on the antibody so as to enhance the ability to detect the presence and amount of the GBS in the sample.

In order to carry out the diagnostic methods of the present invention, it is generally suitable to provide a diagnostic kit may be useful in isolating and identifying GBS bacteria in a patient sample. As would be apparent to one skilled in the art, such kits may generally comprise the antibodies of the present invention in a suitable form, such as lyophilized in a single vessel which then becomes active by addition of an aqueous sample suspected of containing the streptococcal bacteria, along with means to detect binding to the antibodies. Such a kit will typically include a suitable container for housing the antibodies in a suitable form along with a suitable immunodetection reagent which will allow identification of complexes binding to the antibodies of the invention. For example, the immunodetection reagent may comprise a suitable detectable signal or label, such as a biotin or enzyme that produces a detectable color, etc., which normally may be linked to the antibody or which can be utilized in other suitable ways so as to provide a detectable result when the antibody binds to the antigen.

Similarly, a kit in accordance with the invention may also be constructed to detect antibodies to GBS in a sample from a human or animal patient. In such a kit, a suitable amount of FbsA or its active fragments is employed along with means for introducing the patient sample to the protein or fragment, combined with means of detecting whether antibodies in the sample have bound to the FbsA in the kit. Such detection means may include suitable labels and will also allow for the quantification of the GBS antibody titer in the patient.

Accordingly, as indicated above, antibodies in accordance with the invention may be used for the specific detection of GBS infection, for the prevention of adherence of GBS to host cells, for the treatment of an ongoing GBS infection, or for use as research tools. As also indicated above, the term “antibodies” as used herein includes monoclonal, polyclonal, chimeric, single chain, bispecific, simianized, and humanized or primatized antibodies as well as Fab fragments, such as those fragments which maintain the binding specificity of the antibodies to the FbsA protein, including the products of an Fab immunoglobulin expression library. Accordingly, the invention contemplates the use of single chains such as the variable heavy and light chains of the antibodies as will be set forth below. Generation of any of these types of antibodies or antibody fragments is well known to those skilled in the art. In the present case, monoclonal antibodies to FbsA have been generated, and these monoclonal antibodies include monoclonal antibodies such as the one designated 5H2 which were generated against the repeat region of FbsA and which have been shown to block adherence of GBS to fibrinogen and host cells.

Antibodies to FbsA as described above may also be used in production facilities or laboratories to isolate additional quantities of the proteins, such as by affinity chromatography. For example, the antibodies of the invention may also be utilized to isolate additional amounts of the FbsA proteins or their active fragments.

As indicated above, the preferred dose for administration of an antibody composition in accordance with the present invention is that amount will be effective in blocking adherence of GBS to fibrinogen so as to prevent its attachment to host cells, and one would readily recognize that this amount will vary greatly depending on the nature of the infection and the condition of a patient. As indicated above, an “effective amount” of antibody or pharmaceutical agent to be used in accordance with the invention is intended to mean a nontoxic but sufficient amount of the agent, such that the desired prophylactic or therapeutic effect is produced. Thus, the exact amount of the antibody or a particular agent that is required will vary from subject to subject, depending on the species, age, and general condition of the subject, the severity of the condition being treated, the particular carrier or adjuvant being used and its mode of administration, and the like. Accordingly, the “effective amount” of any particular antibody composition will vary based on the particular circumstances, and an appropriate effective amount may be determined in each case of application by one of ordinary skill in the art using only routine experimentation. The dose should be adjusted to suit the individual to whom the composition is administered and will vary with age, weight and metabolism of the individual. The compositions may additionally contain stabilizers or pharmaceutically acceptable preservatives, such as thimerosal (ethyl(2-mercaptobenzoate-S)mercury sodium salt) (Sigma Chemical Company, St. Louis, Mo.).

In short, the antibodies of the present invention which bind to the FbsA will thus be extremely useful in blocking adherence of GBS to host cells, and thus will provide for the treatment and prevention of GBS infections in human and animal patients and in medical or other in-dwelling devices.

In another method in accordance with the present invention, the present inventors have discovered that it is possible to utilize the antibodies of the present invention to prevent or reduce platelet aggregation, and this ability will be useful where, such treatment is necessary, e.g., in preventing or reducing thrombus formation in streptococcal endocarditis and other similar diseases. In the preferred method, a suitable amount of the FbsA antibody or antibody composition is administered to a patient in need of such therapy, and the preferred amount administered is the amount effective to treat or prevent platelet aggregation in a given patient. In this context, “effective amount” once again refers to that nontoxic but sufficient amount of the antibody, such that the desired prophylactic or therapeutic effect is produced with regard to preventing or reducing platelet aggregation. Thus, the exact amount of the antibody that is required will vary from subject to subject, depending on the species, age, and general condition of the subject, the severity of the condition being treated, the particular carrier or adjuvant being used and its mode of administration, and the like. Accordingly, the effective amount will readily be determined by the skilled practitioner in each case based on routine screening of the patient to determine the necessary information with regard to dosage and treatment regimen as adjusted to suit the individual in need of such therapy.

The antibodies of the present invention will thus be very useful in a variety of contexts, most particularly in the area of preventing adherence of GBS to host cells, treating and preventing GBS infections, and in reducing or preventing platelet aggregation in a patient in need of such treatment. Still other features, uses and advantages of the invention will be obtained as described for other MSCRAMM® proteins and/or antibodies thereto, such as those set forth in U.S. Pat. Nos. 5,851,794, 6,288,214, 6,703,025, 6,692,739, 6,685,943 and 6,680,195, all of said patents incorporated herein by reference.

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

### Antibodies to Streptococcus Agalactiae and Their ability to Prevent Platelet Aggregation

Introduction

Streptococcus agalactiae (group B streptococci) is an important human pathogen causing neonatal pneumonia, sepsis, meningitis and severe infections in immunocompromised adult patients. Early-onset neonatal disease is thought to be transmitted by passage of bacteria from colonized mothers to newborns. The first step in the pathogenesis of GBS disease is asymptomatic colonization of the female genital tract. Following maternal colonization, infection reaches the lowest tract of the infant lung airways either trough ascent of bacteria to the amniotic sac or following aspitation of GBS during parturition. Adherence to extracellular matrix components and invasion of pulmonary epithelium may be a prerequisite for infection. In fact, like other pathogens, GBS appear to attach to host extracellular matrix proteins such as fibronectin, laminin and fibrinogen.

Certain evidence (Infect. Immun. 2002, 70, 2408-2413) suggests that C5a peptidase is also a fibronectin-binding protein. Furthermore, a lipoprotein, designated Lmb, has been shown to bind laminin and involved in adherence and invasion of both GBS( Infect. Immun. 1999, 67, 871-878) and S. pyogenes ( Infect. Immun. 2002, 70, 993-997).

The interaction of GBS with fibrinogen has been demonstrated in several studies; however, the molecular basis of fibrinogen binding has remained unknown. Recently, the isolation of the FbsA gene, which encodes a fibrinogen receptor from GBS has been reported ( Mol. Microbiol. 2002, 46, 557-569). The deduced FbsA protein is characterized by repetitive units, each 16 amino acids in length. Sequencing of the FbsA gene from different GBS strains revealed significant variation in the number of the repeat- encoding units. Moreover, using synthetic peptides, even a single repeat unit of FbsA was able to bind to fibrinogen.

### DETAILED DESCRIPTION OF THE EXPERIMENTAL DATA

Adherence of S. agalactiae O90R to fibrinogen.

In preliminary experiments adherence of S. agalactiae O90R to human fibrinogen coated wells was examined. S. agalactiae adhered to fibrinogen in a time-dependent manner (FIG. 1). The kinetics of adherence was relatively slow and attachment process was completed within 90 min. Continuation of the incubation up to 3h did not affect the number of bacteria attached.

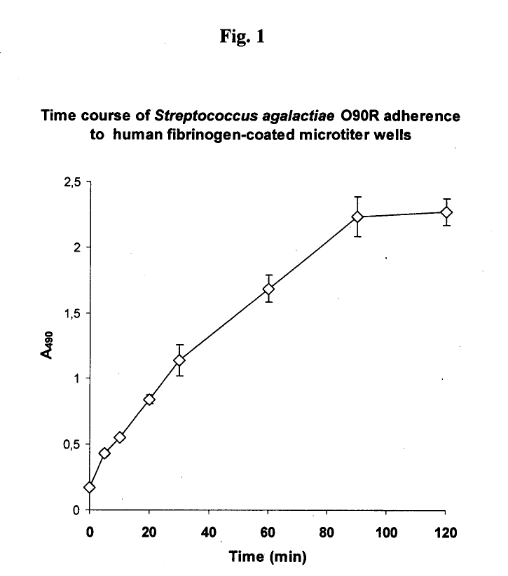

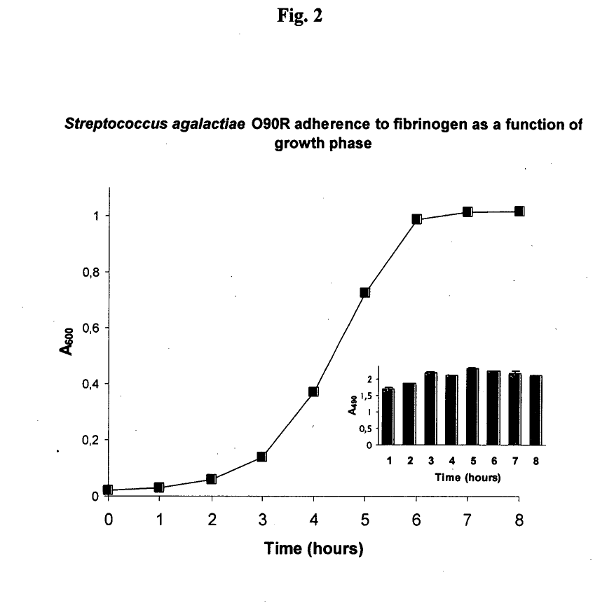

The kinetics of S. agalactiae O90R adherence to fibrinogen as a function of growth phases was monitored culturing bacteria at 37 C. in aerated liquid medium. At each growth phase bacteria were harvested by centrifugation, adjusted to 1×10¹⁰ cells/ml and then assayed for adherence to fibrinogen. As shown in FIG. 2 fibrinogen receptor expression was detectable on cells at all stages of the growth cycle.

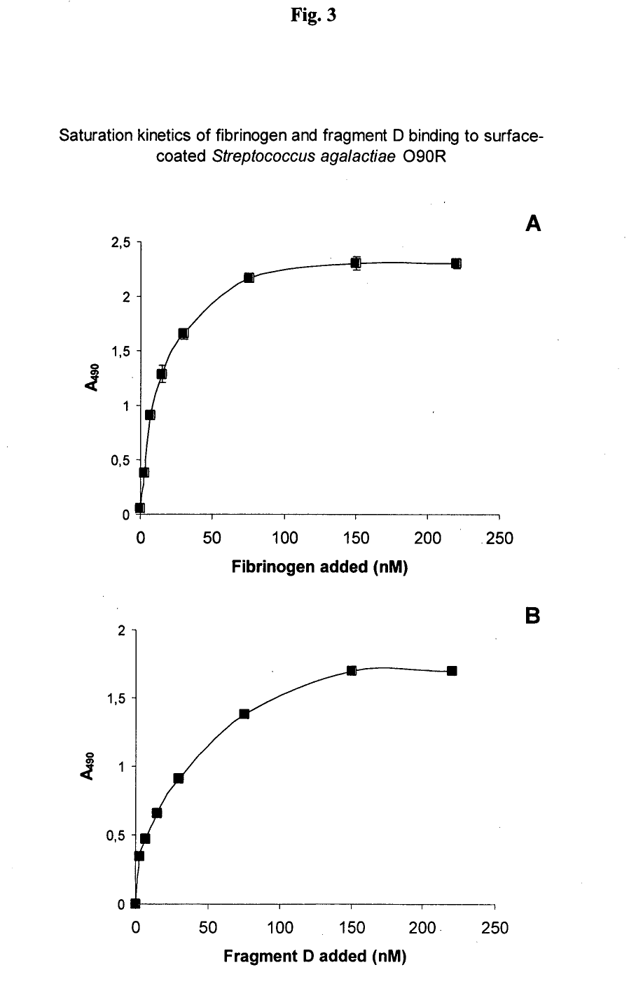

We next examined binding of increasing concentrations of fibrinogen to S. agalactiae cells immobilized onto microtiter wells. In these conditions streptococcal cells were saturated with fibrinogen, suggesting that the interaction involved a limited number of receptors (FIG. 3A). The apparent K_{D} value was estimated from the concentrations of ligand required for half maximal binding. The dissociation constant for fibrinogen was 2.5×10⁻⁸ M.

Cloning and Expression of FbsA from S. agalactiae Strains 6313 and O90R.

FbsA-N, a truncated derivative corresponding to the N-terminal repeat-containing region of FbsA, is an hexahistidine-tagged fusion protein and contains 19 repeat units of 16 amino acid each The protein has been cloned and expressed as reported in Mol. Microbiol. 2002, 46, 557-569.

The FbsA-Binding Site on Fibrinogen.

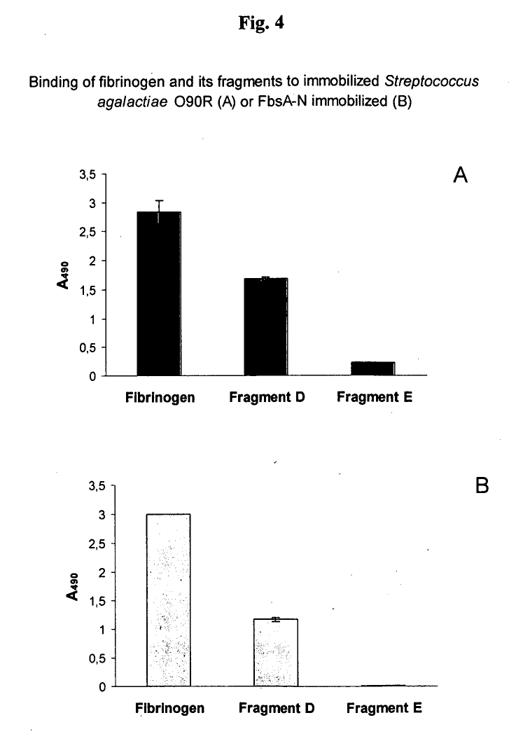

To localize the fbsA-binding site on fibrinogen, we conducted a solid-phase binding assay to determine whether the plasmin generated fibrinogen fragments D or E recognize immobilized purified FbsA-N or intact streptococci. We found that fragment D but not fragment E bound to microtiter wells coated with streptococcal cells (FIG. 4A) or FbsA-N (FIG. 4B) Hence, FbsA-N and bacteria appear to specifically interact with the globular D regions of fibrinogen, mainly consisting of amino acid residues 111-197 of the alpha chain, 134-461 of the beta chain, and 88-406 of the gamma chain. Moreover, fragment D bound microtiter well coated S. agalactiae cells in a concentration-dependent, saturable manner (FIG. 3B). From the saturation kinetics of fragment D binding to bacteria we found a K_{D} value identical to the dissociation constant calculated for fibrinogen binding to streptococci.

Synthesis of pep-FbsA and Generation of Polyclonal anti-pep-FbsA Antibody.

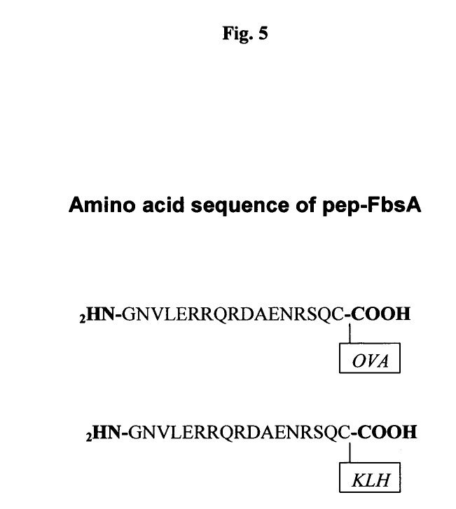

A synthetic peptide corresponding to the repeat unit of FbsA was synthesized by a solid-phase method on a p-benzyloxylbenzylalcohol resin using Fmoc chemistry and a model 350 Multiple Peptide Synthesizer. During the peptide synthesis a cysteine was added to the C-terminal end of the amino acid sequence, that served as anchoring point for coupling ovalbumin(OVA) or keyhole limpet hemocyanin (KLH) (FIG. 5). To produce mouse antisera against the repeat unit of FbsA (anti-pep-FbsA), synthetic peptide coupled to KLH was intraperitoneally injected in BALB/c mice.

Effect of anti-pep-FbsA antibody on Streptococcal Adherence to Fibrinogen.

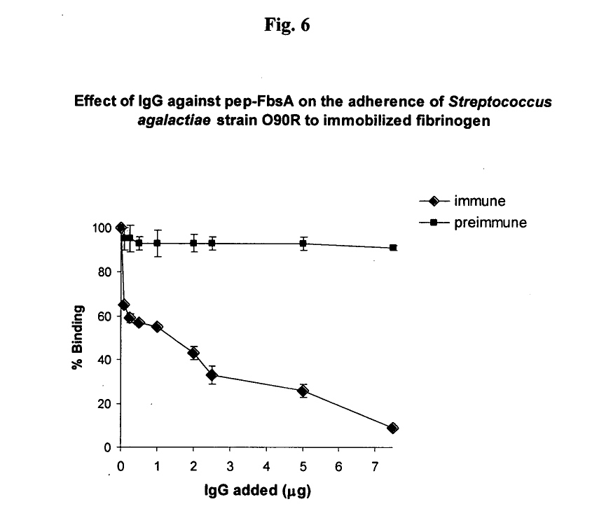

Polyclonal antibodies against pep-FbsA were tested both in ELISA format and Western blot for binding to FbsA-N. In both the cases immune anti-pep-FbsA recognized the recombinant protein. Furthermore, the IgG isolated from the sera inhibited adherence of S. agalactiae O90R to immobilized fibrinogen in a dose-dependent manner, whereas preimmune mouse IgG had no effect (FIG. 6).

Generation of Monoclonal Antibodies Against pep-FbsA and Isotyping.

Pep-FbsA coupled to KLH was also used to produce mAbs: the procedure used BALB/c mice and the mouse myeloma line Spe/0 Ag.14. Hybridoma were screened 10 days postfusion by ELISA for recognition of pep-FbsA coupled to ovalbumin (pep-OVA), and positive hybridomas were rescreened for recognition of the synthetic repeat unit of FbsA. A number of hybridomas that gave a strong ELISA response to pep-OVA were cloned by limiting dilution and the hybridomas 2B1, 5C9, 5H2 and 10H1 were selected and grown to high density in RPMI 1640 medium containing 10% (v/v) fetal bovine serum and antibiotics. The antibody isotype was determined by using the reagent provided with Bio-Rad mouse hybridoma Isotyper Kit. Mabs 5C9, 5H2 and 10H1 were IgG₁₋ₖ and mAb 2B1 was an IgG_{2b-k}.

Binding Specificity of Monoclonal Antibodies Against pep-FbsA.

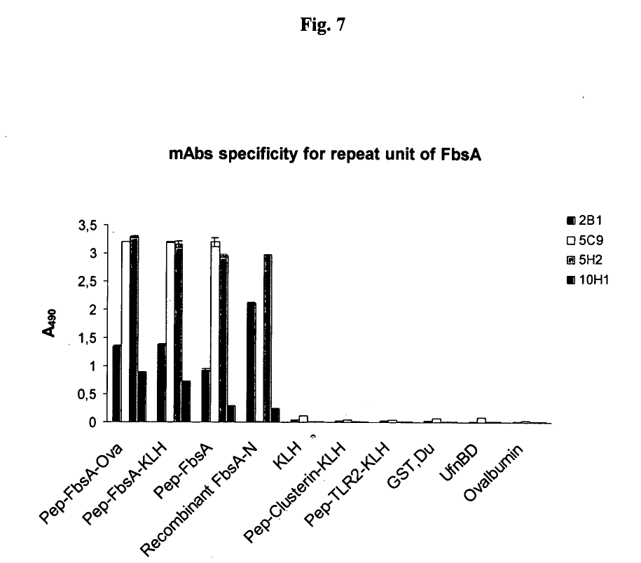

All the mAbs specifically recognized the repeat unit either free or coupled to ovalbumin or KLH. As expected, the antibodies also bound the recombinant FbsA-N. Conversely, no reactivity was observed with synthetic peptide derivatives of similar size from clusterin or Toll Like Receptor 2 (TLR-2) coupled to KLH or other recombinant peptides in fusion with GST (FIG. 7). It is interesting that the monoclonal antibody 5C9 strongly reacts with the synthetic pep-FbsA, but does not show any reactivity with FbsA-N. This finding could be explained if we assume that the single repeat unit may have conformational epitopes that are absent in the full length FbsA.

Effect of mAbs on the Binding of Fibrinogen to FbsA.

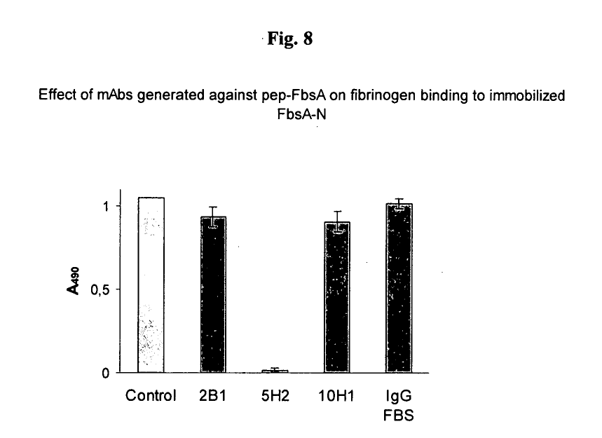

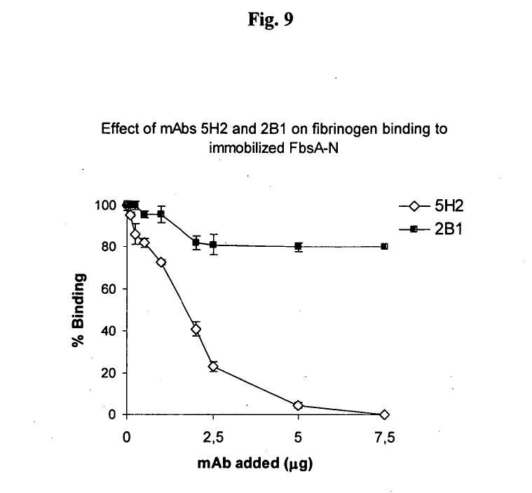

The effect of three mAbs on the binding of fibrinogen to FbsA-N adsorbed in microtiter wells was examined. The mAb 5H2 strongly inhibited fibrinogen binding to FbsA-N. Conversely, the monoclonal antibodies 10H1 and 2B1 or IgG isolated from foetal calf serum were not effective (FIG. 8). The antibody 5H2 also substantially blocked the binding of biotin-labelled fibrinogen to FbsA-N in a concentration-dependent manner (FIG. 9).

Effect of mAbs on the Attachment of Streptococci to Fibrinogen.

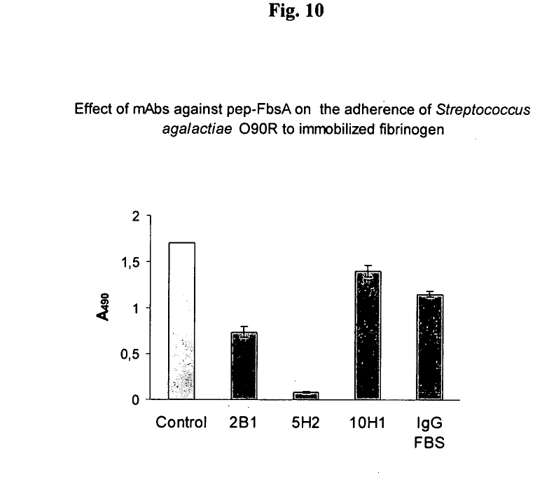

We also examined the effects of mAbs 5H2, 2B1 and 10H1 on adherence of S. agalactiae O90R to fibrinogen. MAb 5H2, which interfered with binding of fibrinogen to FbsA-N, also strongly inhibited the attachment of streptococci to fibrinogen, whereas mAbs B1 and 10H1 had no effect (FIG. 10).

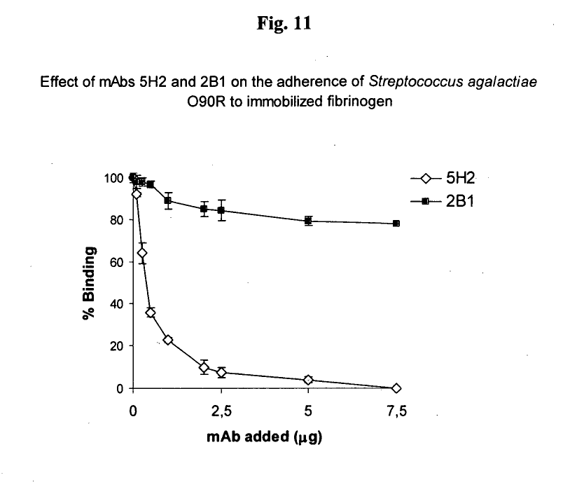

The tendency of concentration-dependent effects on S. agalactiae attachment to fibrinogen by inhibiting 5H2 was further examined and firmly established (FIG. 11).

Streptococcus Agalactiae-Induced Platelet Aggregation.

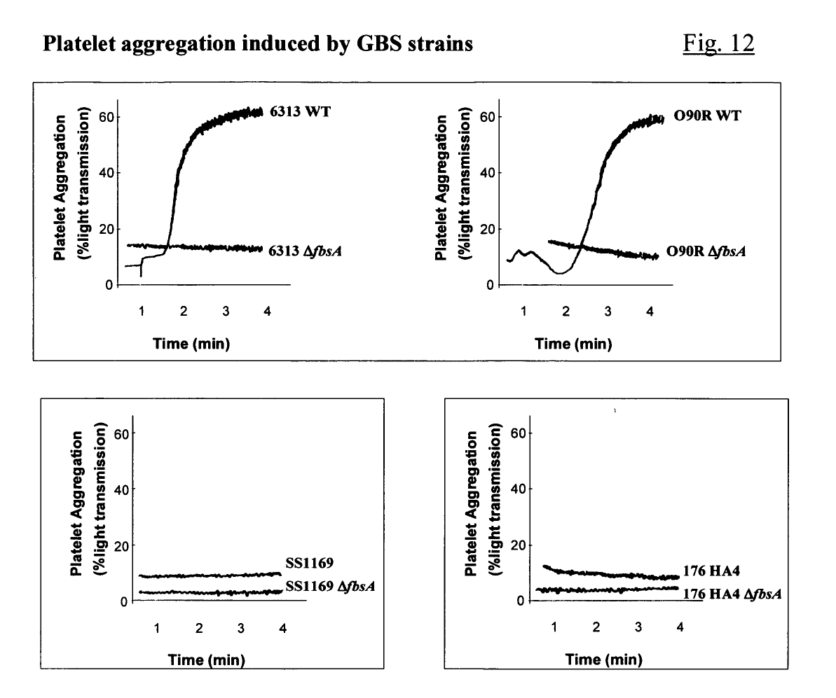

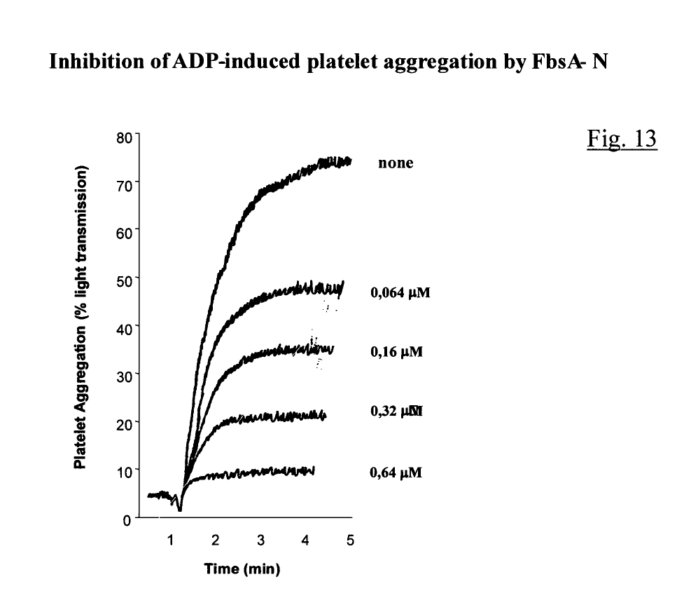

A number of S. agalactiae strains were tested for their ability to induce platelet aggregation and typical examples of traces observed are shown in FIG. 12. Acapsulated streptococci expressing the fibrinogen-binding FbsA (O90R, 6313) supported platelet aggregation. In contrast, the deletion of fbsA gene completely abolished the ability of the strains to aggregate platelets. Streptococcal strains expressing both capsule and a reduced number of FbsA molecules (SS 1169) or displaying FbsA bearing a low number of tandem repeats ( strain 176 HA4, three repeat units) failed also to support platelet aggregation. Together these results support the notion that FbsA represents a critical factor to allow S. agalactiae to promote platelet aggregation. To further confirm the role of FbsA in platelet aggregation, platelet rich plasma was incubated with ADP in the presence of increasing amounts of soluble recombinant FbsA-N (FIG. 13). Expectedly, FbsA inhibited in a dose-dependent manner ADP-induced platelet aggregation. This effect was specific because no influence on the platelet response by other proteins was observed (data not shown).

Streptococcus Agalactiae-Induced Platelet Aggregation is a Genuine Aggregation.

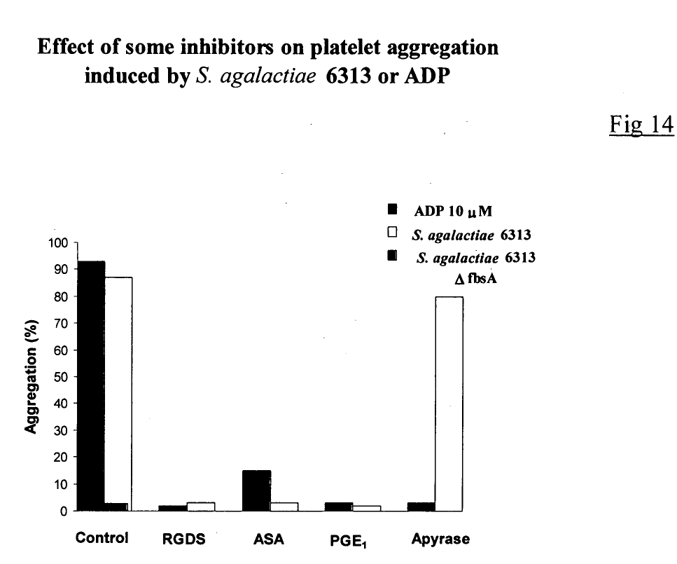

To show that the measure of aggregation observed was reflective of a true platelet activation and is thus genuine aggregation, S. agalactiae-induced aggregation was performed in the presence of specific platelet activation inhibitors (FIG. 14). Preincubation of platelets with PGE₁, which elevates intracellular cAMP thereby inhibiting platelet aggregation, completely inhibited aggregation induced by bacteria, verifying that S. agalactiae cells cause true platelet aggregation. Aggregation was also inhibited by aspirin, a cyclooxygenase inhibitor, suggesting a role for the thromboxane A2 in the aggregation response. However, aggregation was not dependent on the release reaction (some agonists require ADP secretion during activation) because apyrase (ADPase) failed to inhibit 6313-induced aggregation, whereas it completely inhibited ADP-induced platelet aggregation. The finding that GPIIb/IIIa receptor antagonist RGDS also inhibited aggregation of platelets suggests that , although S. agalactiae cells did not bind directly to GPIIb/IIIa, (data not shown), GPIIb/IIIa plays an important role in bacteria-induced platelet aggregation.

Role of Fibrinogen in S. Agalactiae-Induced Platelet Aggregation.

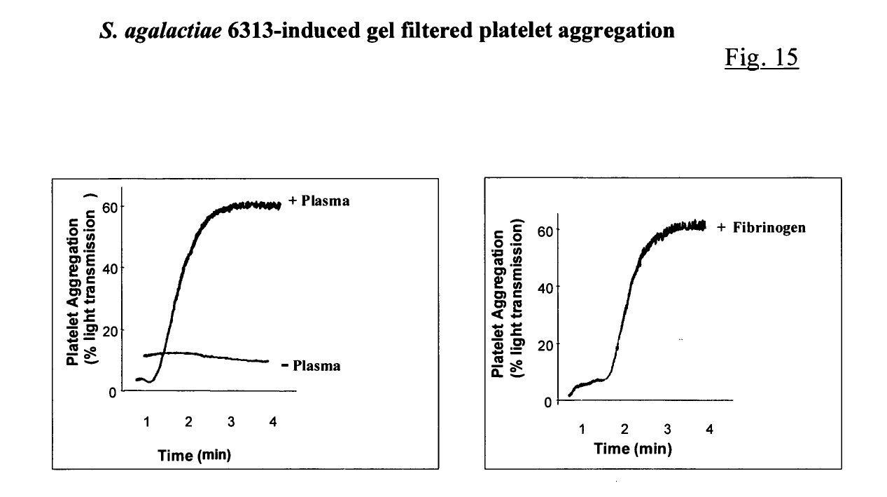

S. agalactiae 6313 induced aggregation of gel filtered platelets in plasma, but not in plasma-free platelets (FIG. 15), suggesting a role for a plasma factor in aggregation. Because fibrinogen is the normal ligand for GPIIb/IIIa and is found in plasma, we investigated its role in S. agalactiae-induced aggregation (FIG. 15). In fact, gel filtered platelets promptly aggregate when incubated with S. agalactiae in the presence of fibrinogen, whereas other plasma protein such as fibronectin did not elicit any platelet response (data not shown).

Effect of anti-pep-FbsA mAb 5H2 on Platelet Aggregation.

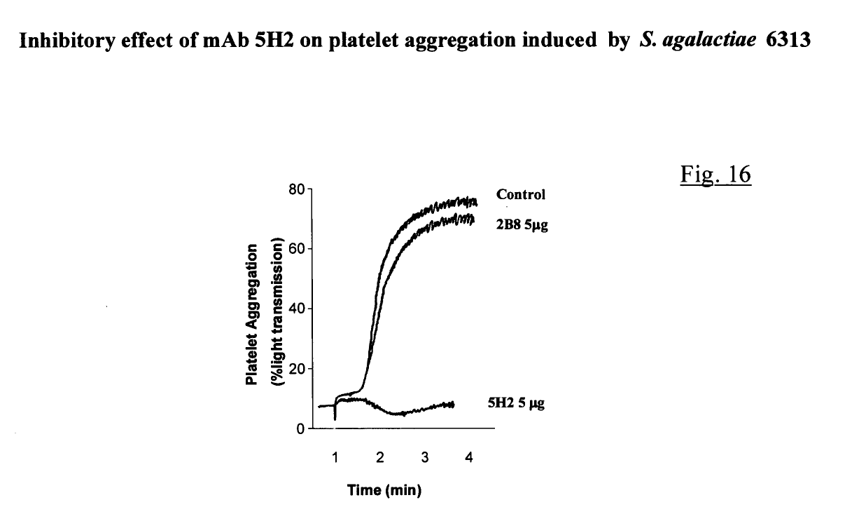

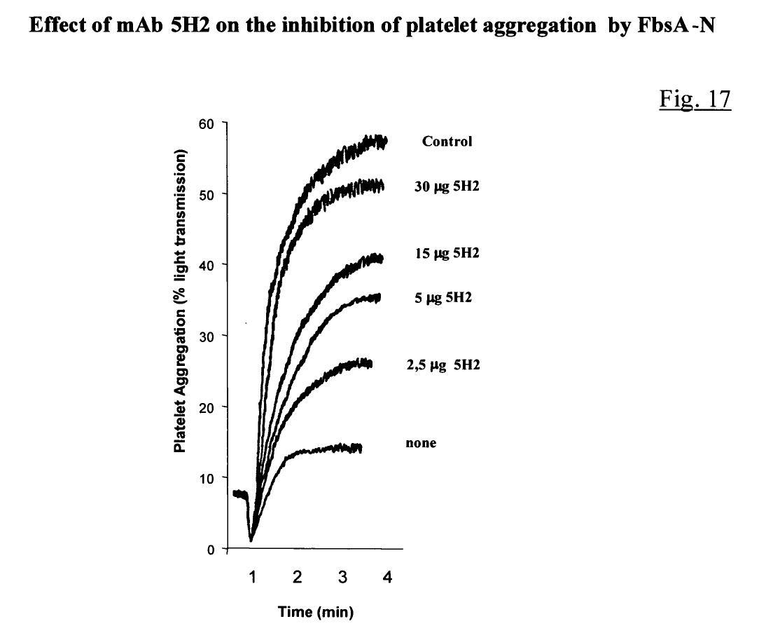

The anti-pep-FbsA mAb 5H2, which blocks adherence of S. agalactiae to fibrinogen, inhibited platelet aggregation, The effect of this antibody was specific because bacteria incubated with isotype-matched mAb 2B8 did not interfere with platelet aggregation (FIG. 16). In addition, 5H2 neutralized in a dose-dependent manner the blocking activity of FbsA-N on ADP-induced platelet aggregation, further confirming the essential role of FbsA in S. agalactiae-induced platelet aggregation. (FIG. 17).

All together these results demonstrate that FbsA in conjunction with fibrinogen contributes to trigger platelet aggregation. More over, we proved the notion that S. agalactiae cells share with S. aureus similar mechanisms in eliciting platelet aggregation, including the binding of platelets to bacteria through adsorbed fibrinogen. Given that the mAb 5H2 effectively blocks bacteria-induced platelet aggregation, it may be worth exploring the therapeutic value of this antibody to combat thrombus formation in the pathogenesis of streptococcal endocarditis.

### Example 2

### Antibodies to FBSA and Their Ability to Prevent the Adherence of Streptococcus Agalactiae to Human Epithelial Cells

Introduction

Streptococcus agalactiae is a major cause of bacterial pneumonia, sepsis and meningitis in human neonates. During the course of infection, S. agalactiae adheres to a variety of epithelial cells but the underlying mechanisms are only poorly understood. The present report demonstrates the importance of the fibrinogen-receptor FbsA for the streptococcal adherence and invasion of epithelial cells. Deletion of the fbsA gene in various S. agalactiae strains substantially reduced their fibrinogen-binding, and their adherence and invasion of epithelial cells, indicating a role of FbsA in these different processes. The adherence and invasiveness of an fbsA deletion mutant was partially restored by re-introducing the fbsA gene on an expression vector. Heterologous expression of fbsA in Lactococcus lactis allowed the bacteria the adherence but not the invasion of epithelial cells, suggesting that FbsA is a streptococcal adhesin. Flow cytometry experiments revealed a dose-dependent binding of FbsA to the surface of epithelial cells. Furthermore, tissue culture experiments exhibited an intimate contact of FbsA-coated latex beads with the surface of human epithelial cells. Finally, host cell adherence and invasion was significantly blocked in competition experiments with either purified FbsA protein or a monoclonal antibody, directed against the fibrinogen-binding domain of FbsA. Taken together, our studies unambiguously demonstrate FbsA-mediated adherence of S. agalactiae to epithelial cells. Our findings also indicate, that adherence of S. agalactiae is a prerequisite for subsequent bacterial entry into host cells, and that fibrinogen-binding domains within FbsA are also involved in host cell adherence.

Background

Streptococcus agalactiae is a frequent colonizer of the gastrointestinal and urogenital tract of humans (3). However, it is also the cause of substantial pregnancy-related morbidity and has emerged as an increasingly common cause of invasive disease in the elderly and in immunocompromised persons (55). In addition, S. agalactiae is the most common cause of bacterial pneumonia, sepsis and meningitis in human newborns (3). Neonates acquire S. agalactiae from colonized mothers by aspiration of infected amniotic fluid or vaginal secretions at birth, followed by bacterial adherence to pulmonary epithelial cells (47). Adherence of the bacteria to lung epithelial cells is a prerequisite for the invasion of deeper tissues and the dissemination of the bacteria to the bloodstream. Several studies have demonstrated the adherence of S. agalactiae to epithelial cells both in vitro and in vivo (5, 42, 49, 61). However, the underlying mechanisms of this interaction are only poorly understood. Lipoteichoic acid (LTA) was initially postulated to mediate the adherence of S. agalactiae to epithelial cells (32, 33) but later studies demonstrated a cytotoxic effect of LTA on eukaryotic cells (15). As pretreatment of S. agalactiae with protease decreases the bacterial adherence to host cells (31, 49), surface proteins are presently assumed to be important for this process. However, the bacterial determinants that promote adherence of S. agalactiae to epithelial cells have not been elucidated. Numerous pathogenic bacteria adhere to host cells by surface proteins, termed adhesins, that bind to components of the extracellular matrix (ECM). The ECM of mammalian tissues consists of glycoproteins, including collagen, laminin, fibronectin and fibrinogen, which form a macromolecular structure underlying epithelial and endothelial cells (21). Several studies have described interactions of S. agalactiae with the ECM proteins laminin, fibronectin, and fibrinogen (26, 48, 51). For each of these binding functions, corresponding bacterial receptors have been identified. In S. agalactiae, the C5a peptidase was shown to play a role in fibronectin-binding (4), and the protein Lmb mediates binding to human laminin (48). Recently, we identified in S. agalactiae two fibrinogen-binding proteins, termed FbsA and FbsB, respectively (19, 44). On the amino acid level, FbsA and FbsB are unrelated to each other, but they have a surface-exposed localization in the cell wall of the bacteria. The FbsB protein was shown to bind to human fibrinogen by its N-terminal 388 amino acids (19) whereas the FbsA protein interacts with fibrinogen by repetitive units, each 16 amino acids in length (44). Even a single repeat of FbsA was demonstrated to bind to human fibrinogen (44). Epidemiological studies revealed significant variation in the number of repeats in the FbsA protein between various S. agalactiae strains. Thus, FbsA variants ranging between three and thirty repeats have been described in different clinical isolates. The FbsA protein was already shown to protect the bacteria from opsonophagocytosis, indicating a role of this protein for the virulence of S. agalactiae.

The present study investigates the importance of FbsA in the adherence and invasion of epithelial cells by S. agalactiae. Defined fbsA deletion mutants were constructed and tested for their interaction with host cells. The effect of plasmid-mediated fbsA expression on bacterial cell adherence and invasion was tested both in S. agalactiae and in Lactococcus lactis. Furthermore, flow cytometry and latex beads experiments were performed to analyze the interaction of FbsA with the surface of epithelial cells. Finally, we tested the influence of FbsA protein and of FbsA-specific monoclonal antibodies on host cell adherence and invasion by S. agalactiae.

Materials and Methods

Bacterial Strains, Epithelial Cells and Growth Conditions.

The S. agalactiae strains 6313 (serotype III), 706 S2 (serotype Ia), O176 H4A (serotype II), and SS1169 (serotype V) are clinical isolates and have been described previously (44). S. agalactiae strain 6313 ΔfbsA is an fbsA deletion mutant of strain 6313 (44) and strain O90R (ATCC 12386) is a capsule mutant of the serotype Ia strain O90. S. agalactiae was cultivated at 37° C. in Todd-Hewitt yeast broth (THY) containing 1 % yeast extract. S. agalactiae strains carrying the plasmids pOri23 or pOrifbsA were grown in the presence of erythromycin (5 μg/ml). E. coli DH5α (20) was used for cloning purposes and E. coli BL21 (12) served as host for the production of FbsA fusion protein. E. coli was grown at 37° C. in Luria broth (LB) and clones carrying pOri23- or pET28-derivatives (44) or the plasmid pG⁺ΔfbsA (44), were selected in the presence of erythromycin (300 μg/ml), kanamycin (50 μg/ml) or ampicillin (100 μg/ml). Lactococcus lactis subsp. cremoris MG1363 (14) was used for heterologous expression of the fbsA gene. L. lactis was grown at 30° C. in M17 medium (Oxoid), supplemented with 0.5% glucose, and strains carrying pOri23 or pOrifbsA were selected with 5 μg/ml erythromycin.

The cell line A549 (ATCC CCL-185) was obtained from the American Type Culture Collection. A549 is a human lung carcinoma cells which has many characteristics of type I alveolar pneumocytes. A549 cells were propagated in RPMI tissue culture medium (Gibco BRL) with 10% of fetal calf serum in a humid atmosphere at 37° C. with 5% CO₂.

Construction of fbsA Deletion Mutants in S. agalactiae.

The fbsA gene was deleted in the S. agalactiae strains O90R, 706 S2, O176 H4A, and SS1169 according to the procedure described by Schubert et al. (44). Briefly, the thermosensitive plasmid pG⁺ΔfbsA was transformed into the S. agalactiae strains by electroporation and transformants were selected by growth on erythromycin agar at 30° C. Cells in which pG⁺ΔfbsA had integrated into the chromosome were selected by growth of the transformants at 37° C. with erythromycin selection as described (27). Integrant strains were serially passaged for five days in liquid medium at 30° C. without erythromycin selection to facilitate the excision of plasmid pG+ΔfbsA, leaving the desired fbsA deletion in the chromosome. Dilutions of the serially passaged cultures were plated onto agar and single colonies were tested for erythromycin sensitivity to identify pG⁺ΔfbsA excisants. Chromosomal DNA of erythromycin sensitive S. agalactiae excisants was tested by Southern blot after HindIII digestion using a digoxigenin-labelled fbsA flanking fragment as described (44).

Plasmid-mediated Expression of fbsA in S. agalactiae and L. lactis.

The fbsA structural gene, including its ribosomal binding site, was amplified from chromosomal S. agalactiae 6313 DNA by PCR using the primers 5′GTTTAGTGGATCCGAAGTAAGGAGAAAATTAATTGTTC (SEQ ID NO:1) and 5′ATCCCATATAATGACCTC (SEQ ID NO:2), and the PCR product was directly ligated into the T/A cloning vector pDrive (Qiagen). The fbsA gene was subsequently isolated by BamHI digest and ligated into the BamHI digested E. coli/Streptococcus expression vector pOri23 (40). The orientation of the fbsA gene in pOri23 was determined by HindIII digest, and the resulting plasmid was termed pOrifbsA. Vector pOri23 and plasmid pOrifbsA were transformed by electroporation into S. agalactiae and L. lactis with subsequent erythromycin selection. L. lactis cells were made competent and transformed as described elsewhere (57).

Antibodies and Human Proteins.

Affinity-purified rabbit anti-fibrinogen antibodies were obtained from Dako-Biochemicals. Fibrinogen (Sigma) was passed through a gelatin-Sepharose column to remove residual contaminating fibronectin in the preparation. The purity of the fibrinogen preparation was confirmed by SDS-PAGE and Coomassie-staining and by Western blotting using anti-fibronectin antibodies (Sigma-Aldrich). The generation and characterization of the anti FbsA monoclonal antibodies 5H2 and 2B1 will be described elsewhere (Pietrocola et al., manuscript in preparation).

Binding of Soluble ¹²⁵I-labelled Fibrinogen to S. agalactiae

Purified human fibrinogen was radiolabelled with ¹²⁵I, using the chloramin T method (22). Binding of labelled fibrinogen to S. agalactiae was performed as described previously (44).

Preparation of Hexahistidyl-Tagged Fusion Proteins.

The FbsA fusion protein originates from S. agalactiae 6313 and possesses 19 repeats, each 16 amino acids in length (44). The Bsp protein is a surface protein from S. agalactiae that plays a role in the morphogenesis of the bacteria (41) and served as a control in the present study. The fusion proteins were synthesized in recombinant E. coli BL21 by the addition of 1 mM IPTG after the culture had reached an optical density of 1.0. The cells were disrupted using a French Press cell and purification of the fusion protein was performed according to the instructions of Qiagen using Ni²⁺ affinity chromatography.

Adherence and Invasion Assays.

Adherence of S. agalactiae to epithelial cells and internalization into epithelial cells was assayed as described previously (18). Briefly, A549 cells were transferred to 24-well tissue culture plates at approximately 4×10⁵ cells per well and cultivated overnight in RPMI tissue culture medium, supplemented with 10% of fetal calf serum. After replacement of the medium with 1 ml of fresh medium, the cells were infected with S. agalactiae at a multiplicity of infection (MOI) of 10:1, and incubated at 37° C. for 2 h. The infected cells were subsequently washed three times with phosphate-buffered saline (PBS). The number of cell-adherent bacteria was determined by lysis of the eukaryotic cells with distilled water and subsequent determination of colony-forming units (cfu) by plating appropriate dilutions of the lysates on THY agar. Intracellular bacteria were determined after a further incubation of the infected cells for 2 h with RPMI medium containing penicillin G (10 U) and streptomycin (0.01 mg) to kill extracellular bacteria. After three washes with PBS, the epithelial cells were lysed in distilled water and the amount of intracellular bacteria was quantitated by plating serial dilutions of the lysate onto THY agar plates. All samples were tested in triplicate, and experiments were repeated at least three times.

To assess the effect of FbsA, Bsp or polyclonal anti fibrinogen antibodies on the adherence and invasion of S. agalactiae, the adherence and invasion assays were performed as described above, with the following modifications: A549 cells in tissue culture wells were incubated for 15 min in 100 μl of PBS with different amounts of purified proteins or antibodies as described elsewhere (29). Bacterial cells were then added in tissue culture medium and the wells were incubated at 37° C. for 2 h. To analyze the effect of anti FbsA monoclonal antibodies on the bacterial adherence and invasion, S. agalactiae 6313 was incubated for 15 min in 500 μl of RPMI medium, containing different amounts of the monoclonal antibodies. Subsequently, the bacteria were used to infect A549 cells and the remainder of the experiment was carried out as described above.

FACS Analysis.

Binding of purified FbsA protein to A549 cells was performed essentially as described by Taschner et al. (52). In brief, 5×10⁶ A549 cells were pelleted by centrifugation at 4° C. and washed with 10% BSA in PBS. Subsequently, the cells were incubated for 45 min on ice with 5 μg of Fc fragments (Dianova), and washed two times with 10% BSA in PBS. The cells were incubated for 1 h with different concentrations of FbsA fusion protein on ice, again washed two times with 10% BSA in PBS, and incubated for 1 h on ice with a monoclonal anti-His-tag antibody (1:100; Qiagen). After two washings with 10% BSA in PBS, FITC-labelled anti-mouse IgGs (1:500; Dako) were added and the suspension was incubated for 1 h on ice. The cells were again washed two times with 10% BSA in PBS and fixed for 30 min with 1% paraformaldehyde in PBS. The fluorescence of 10⁵ cells was quantitated in a FACSCalibur flow cytometer (Becton Dickinson) and the data were analyzed with the WinMDI software.

Scanning Electron Microscopy of FbsA-coated Latex Beads.

Approximately 1×10⁹ latex beads (3 μm diameter, Sigma) were washed three times in 25 mM 2-N-morpholinoetanesulfonic acid (MES), pH 6.8. One half was resuspended in 1.0 ml MES buffer containing 500 μg/ml FbsA fusion protein and the remaining half was resuspended in 1.0 ml MES buffer with 100 mg/ml BSA. The beads were incubated overnight at 4° C. with end-over-end rotation. After pelleting of the beads by centrifugation, the amount of remaining protein in the supernatant was determined with a Bradford protein assay kit (BioRad). The beads were washed once with MES buffer and blocked for 1 h with 10 mg/ml BSA in MES buffer at room temperature. The beads were washed twice with MES buffer, once with RPMI+10% FCS, and resuspended in RPMI+10% FCS. Confluent A549 cells in 24-well plates were inoculated with 2×10⁸ beads per well in a total volume of 1.0 ml. The bead monolayer mixtures were incubated for 2 h at 37° C. in a 5% CO₂ atmosphere. Cells were washed five times with PBS and fixed with 3% paraformaldehyde and 4% glutaraldehyde in 0.1% cacodylate buffer for scanning electron microscopy. Scanning electron microscopy was performed with a Zeiss DSM 962 microscope.

Results

Various S. agalactiae Strains Require the fbsA Gene for Fibrinogen-Binding.

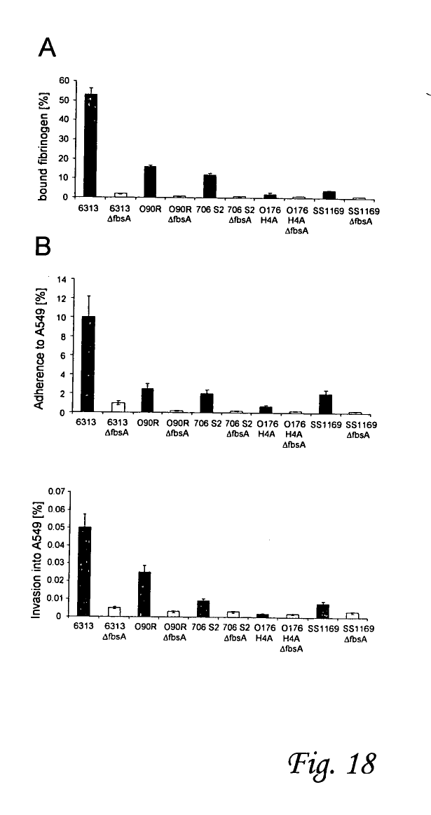

In the clinical S. agalactiae isolate 6313 (serotype III), the FbsA protein is essential for the attachment to human fibrinogen (44). To analyze the importance of FbsA for the fibrinogen-binding of various S. agalactiae strains, the fbsA gene was deleted in the genomes of S. agalactiae 706 S2 (serotype Ia), 0176 H4A (serotype II), and SS1169 (serotype V). The fbsA gene was also deleted in the chromosome of S. agalactiae O90R, a capsule mutant of the serotype Ia strain O90. By Southern blot analysis the successful deletion of fbsA in the genome of the respective strains was confirmed (data not shown). The different S. agalactiae strains and their fbsA mutants were subsequently tested for their binding of ¹²⁵ I-labelled fibrinogen (FIG. 18A). S. agalactiae 6313 revealed significant binding of radiolabelled fibrinogen whereas the strains O90R and 706 S2 exhibited moderate, and strains SS1169 and O176 H4A only little binding of soluble fibrinogen. In all of the tested strains, however, the deletion of fbsA resulted in a loss of their fibrinogen-binding activity, indicating that the fbsA gene is essential for the fibrinogen-binding of various S. agalactiae strains.

S. agalactiae Host Cell Adherence and Invasion is FbsA-Dependent.

To investigate the importance of FbsA for host cell adherence and invasion of S. agalactiae, the strains 6313, O90R, 706 S2, O176 H4A and 1169, and their isogenic fbsA mutants were used in tissue culture experiments with the human lung epithelial cell line A549. As shown in FIG. 18B, strain 6313 efficiently adhered to and invaded A549 cells, whereas strains O90R, 706 S2 and SS1169 revealed a moderate, and strain O176 H4A a low adherence and invasion of A549 cells. Interestingly, the capability of the various strains for host cell adherence and invasion correlated with their ability for fibrinogen-binding, indicating a putative connection between fibrinogen-binding and host cell interaction in S. agalactiae. In line with this, the deletion of the fbsA gene substantially reduced the host cell adherence and invasion of the different S. agalactiae strains. Only the invasiveness of strain O176 H4A, being already very low, was not further reduced upon deleting the fbsA gene. Our findings therefore indicate a prominent role of the fibrinogen-binding protein FbsA in the adherence and invasion of epithelial cells by S. agalactiae. To study the importance of FbsA for host cell adherence and invasion of S. agalactiae in more detail, we focussed on the highly adherent and invasive S. agalactiae strain 6313.

Plasmid-Mediated Expression of fbsA Partially Restores Host Cell Adherence and Invasion of S. agalactiae 6313 ΔfbsA.

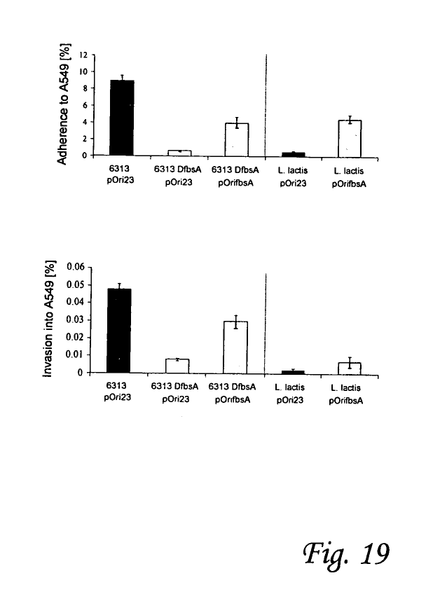

To complement the fbsA deficiency of mutant 6313 ΔfbsA, we attempted to clone from strain 6313 the entire fbsA gene, including its promotor region, into the E. coli/Streptococcus shuttle vector pAT28. Despite several attempts, we repeatedly failed to clone the fbsA gene into this vector. As the promotor of the fbsA gene is very active both in E. coli and S. agalactiae ((18) and unpublished results), we hypothesized that overexpression of fbsA by its own promotor might be toxic to E. coli and S. agalactiae. We therefore cloned the promotorless fbsA gene into the E. coli/Streptococcus expression vector pOri23 (Que et al., 2000), resulting in plasmid pOrifbsA. S. agalactiae 6313 and 6313 ΔfbsA were transformed with the plasmids pOri23 and pOrifbsA, respectively, and subsequently examined for their adhesive and invasive capacity for A549 cells (FIG. 19). The S. agalactiae strains 6313 pOri23 and 6313 ΔfbsA pOri23 revealed comparable adherence and invasion rates as their plasmid-free parental strains, demonstrating that the vector pOri23 does not influence the adherence and invasion properties of these strains. In contrast, plasmid-mediated expression of fbsA in strain 6313 ΔfbsA pOrifbsA significantly increased its adherence and invasion of A549 cells compared to strain 6313 ΔfbsA pOri23. Our findings therefore demonstrate that the reduced adherence and invasion of A549 cells by mutant 6313 ΔfbsA is due to its fbsA deficiency and not to unrelated mutations in its chromosome. However, the adhesive and invasive efficiency of 6313 ΔfbsA pOrifbsA was significantly lower than that of 6313 pOri23, indicating that pOri23-driven expression of fbsA does not to fully complement the fbsA deficiency of mutant 6313 ΔfbsA.

Heterologous Expression of fbsA in Lactococcus Lactis Confers the Ability for Host Cell Adherence.

To investigate whether S. agalactiae factors other than FbsA are required for the bacterial adherence and invasion of host cells, the plasmids pOri23 and pOrifbsA were introduced in L. lactis, a Gram-positive bacterium that naturally does not adhere to epithelial cells. The resulting transformants were subsequently examined for their adhesive and invasive capacity with A549 cells (FIG. 19). L. lactis pOri23 exhibited no adherence and invasion of A549 cells whereas L. lactis pOrifbsA showed significant adherence to A549 cells but only little invasion into this cell line. Of note, host cell adherence of L. lactis pOrifbsA was in the same magnitude of order as that of the complemented S. agalactiae strain 6313 □fbsA pOrifbsA (FIG. 19). In contrast, the invasiveness of L. lactis pOrifbsA was as low as that of the fbsA deletion mutant. These findings demonstrate that FbsA does not require an S. agalactiae co-receptor for host cell adherence. Our results also indicate that the FbsA protein promotes the bacterial adherence but not the invasion into host cells.

The FbsA Protein Binds Directly to A549 Cells.

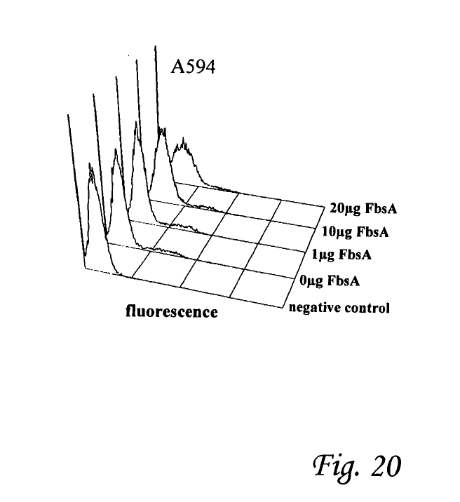

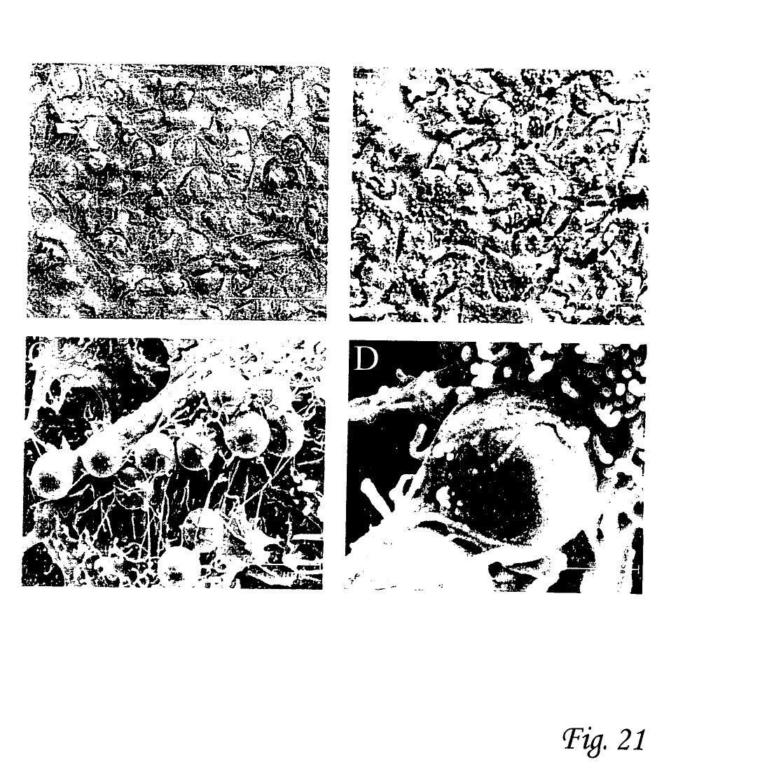

Flow cytometry and latex beads experiments were performed to investigate the interaction of FbsA with A549 cells in more detail. In flow cytometry experiments, a dose-dependent binding of the FbsA fusion protein to A549 cells was observed (FIG. 20), suggesting that FbsA binds directly to host cells. To further investigate the interaction of FbsA with epithelial cells, latex beads were coated with the FbsA fusion protein and tested for their interaction with human A549 cells. As a control, bovine serum albumin (BSA)-coated latex beads were analyzed for their binding to A549 cells. By scanning electron microscopy, BSA-coated latex beads were rarely found associated with A549 cells (FIG. 21A), while FbsA-coated beads bound in high numbers to A549 cells (FIG. 21B). Attachment of the FbsA-coated beads to the plasma membrane of A549 cells was characterized by contact with microvilli and structures that resembled early pseudopod formation (FIG. 21C). In a few cases, the pseudopod appeared to surround the surface of the bead, indicating that the bead was finally internalized (FIG. 21D). Taken together, the results from our flow cytometry and latex beads experiments indicate a direct interaction of FbsA with structures on the surface of A549 cells.

The FbsA Protein Blocks the Bacterial Adherence and Invasion of A549 Cells.

As the previous experiments had demonstrated direct binding of FbsA to the surface of epithelial cells, we investigated the effect of externally added FbsA fusion protein on the adherence and invasion of A549 cells by S. agalactiae. Pretreatment of A549 cells with 50 μg/ml of FbsA fusion protein reduced the adherence of S. agalactiae 6313 by 51±6% and its invasion by 46±7%. Similarly, preincubation of A549 cells with 100 μg/ml of FbsA inhibited the adherence of strain 6313 by 71±5% and its invasion by 73±7%. Pretreatment of A549 cells with 100 μg/ml of the S. agalactiae protein Bsp, which plays a role in the morphogenesis of the bacteria (41), did not influence the bacterial adherence and invasion of A549 cells (data not shown). These results demonstrate that externally added FbsA protein can specifically block host cell adherence and invasion of S. agalactiae.

A Monoclonal Antibody Against the Fibrinogen-Binding Site of FbsA Blocks the Bacterial Adherence.

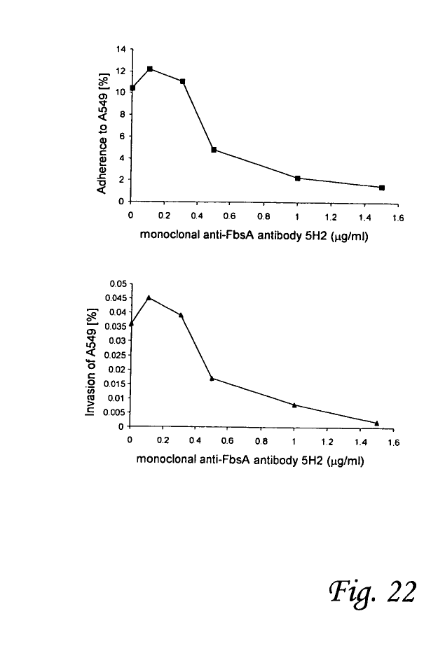

To better understand the interaction of FbsA with the host cell surface on the molecular level, we used monoclonal antibodies directed against different regions of the FbsA protein (Pietrocola et al., manuscript in preparation). Monoclonal antibody 5H2 (mAb 5H2) binds to the repeat region of FbsA, thereby blocking the fibrinogen-binding of the FbsA protein. In contrast, monoclonal antibody 2B1 (mAb 2B1) binds to the repeat region of FbsA without interfering with the binding of FbsA to human fibrinogen. After preincubating S. agalactiae 6313 with either of the two monoclonal antibodies, the streptococcal host cell adherence and invasion was quantitated in tissue culture experiments. As depicted in FIG. 22, increasing concentrations of mAb 5H2 caused a dose-dependent inhibition of the bacterial adherence and invasiveness. Preincubation of strain 6313 with 1.5 μg/ml of mAb 5H2 almost completely blocked the streptococcal adherence and invasion of A549 cells. In contrast, preincubation of strain 6313 with even 10 μg/ml of mAb 2B1 did not influence its host cell adherence or invasion (data not shown).

Tissue culture experiments were performed to analyze the importance of host cell fibrinogen for the streptococcal adherence and invasion of epithelial cells. Pretreatment of strain 6313 with human fibrinogen caused a dose-dependent inhibition of the adherence and invasion of epithelial cells (data not shown) but it also resulted in the previously described clumping of the bacteria (19). We therefore tested the effect of polyclonal anti-fibrinogen antibodies on the adherence and invasion of A549 cells by S. agalactiae. Pretreatment of A549 cells with up to 200 μg/ml of polyclonal anti fibrinogen antibodies did not influence the adherence and invasiveness of strain 6313 (data not shown). However, the antibodies did neither interfere with the binding of S. agalactiae to human fibrinogen (data not shown).

Discussion

The adherence of streptococci to epithelial cells is a key event in the infection process that allows the colonization of host epithelial surfaces (47). Following colonization, the bacteria may eventually penetrate the epithelial barrier and disseminate to the bloodstream and deeper tissues. Adherence is frequently mediated by specific interactions between streptococcal cell wall proteins and components of the host extracellular matrix (ECM). Several studies have demonstrated the presence of fibrinogen in the ECM of human lung epithelial cells (16, 17, 34). S. agalactiae, a frequent cause of neonatal pneumonia, was recently shown to synthesize the fibrinogen-binding proteins FbsA and FbsB (19, 44). The present study was aimed to investigate the importance of FbsA for the binding of S. agalactiae to human fibrinogen, and for the bacterial adherence and invasion of epithelial cells.

Previously, the fbsA gene was found to be widely distributed in different S. agalactiae strains, and to be essential for the fibrinogen-binding of S. agalactiae 6313 (44). However, the importance of FbsA for the fibrinogen-binding of various clinical S. agalactiae isolates remained unclear. Here, we provide evidence that FbsA represents the major fibrinogen receptor in various S. agalactiae strains, belonging to different serotypes. This suggests that FbsA is of general importance for the fibrinogen-binding of S. agalactiae. Interestingly, the fbsB gene, encoding the second fibrinogen-binding protein in S. agalactiae, was found not to influence fibrinogen-binding (19), supporting the hypothesis of FbsA being the major fibrinogen-binding protein in S. agalactiae. Also Staphylococcus aureus and S. pyogenes possess different fibrinogen-binding-proteins (9, 58). (24, 30, 35, 37, 56)In S. pyogenes, fibrinogen-binding is predominantly mediated by M-proteins (10, 23, 25, 38, 39, 54) whereas S. aureus interacts with fibrinogen growth-phase dependently by ClfA or ClfB (30, 35). These data indicate that different streptococcal and staphylococcal species possess one major fibrinogen-receptor in parallel with accessory fibrinogen-binding proteins.

Of note, the S. agalactiae strains used in the present study revealed significant differences in their ability to interact with human fibrinogen. Recently, the internal repeats of the highly repetitive FbsA protein were shown to mediate fibrinogen-binding, and even a single repeat of FbsA was demonstrated to interact with fibrinogen (44). In our study, the FbsA proteins of S. agalactiae 6313, O90R, 706 S2, 176 H4A and SS1169 differed from each other in that they possess 19, 10, 17, 3, and 30 internal repeats. Interestingly, strain SS1169, whose FbsA protein carries 30 internal repeats, revealed only weak fibrinogen-binding. Similarly, strain O90R, synthesizing an FbsA protein with 10 internal repeats, bound higher amounts of fibrinogen than strain 706 S2, whose FbsA protein carries 17 repetitive units. These findings do not indicate a correlation between the repeat number of the FbsA protein and the fibrinogen binding capability of a given strain. Possibly, the analyzed strains differ in respect to their fbsA expression, the transport of the FbsA protein across the cytoplasmic membrane or the FbsA anchoring in the cell wall. Alternatively, the capsule of the different strains may influence their fibrinogen-binding properties. In a report by Chhatwal et al. (8), the capsule of S. agalactiae was demonstrated to interfere with the bacterial binding to fibrinogen. Studies are therefore underway to investigate in the different strains the expression of the fbsA gene and the importance of the capsule for fibrinogen-binding.

The initial event in the colonization of host surfaces by S. agalactiae is the adherence of the bacteria to epithelial surfaces which involves specific interactions between bacterial adhesins and host receptors. In various in vitro models, S. agalactiae was shown to adhere to vaginal epithelial cells (6, 28, 49, 53, 61), buccal epithelial cells (2, 5, 53, 59), chorion and amnion epithelial cells (60), and pulmonary epithelial cells (7, 49, 50). However, the molecular basis for host cell adherence of S. agalactiae was only poorly understood. The laminin-binding protein Lmb is speculated to play a role in the colonization of epithelial surfaces (47) but this has not been experimentally tested. Recently, the transcriptional regulator RogB and the oligopeptide permease Opp were shown to control the adherence of S. agalactiae to epithelial cells as well as FbsA-mediated fibrinogen-binding (18, 43). These findings indicated a link between the fibrinogen-receptor FbsA and the adherence of S. agalactiae to epithelial cells. In the present study, different experimental approaches unambiguously demonstrate, that FbsA is sufficient to promote the adherence of S. agalactiae to epithelial cell. Thus, FbsA represents the first identified adhesin in these bacteria.

As revealed by competition experiments and the analysis of fbsA deletion mutants, the invasion of epithelial cells by S. agalactiae is clearly dependent on the FbsA protein. This might indicate that FbsA is not only an adhesin but also an invasin in S. agalactiae. However, adherence is frequently a prerequisite for the successful invasion of host cells (13). In line with this, the adherence of the fbsA deletion mutants was reduced by the same order of magnitude as it was their host cell invasion. Similar results were obtained in the competition experiments using purified FbsA protein or mAb 5H2. Calculation of the internalization index, which relates the invasion of the bacteria to their adherence (13) shows no difference between the fbsA mutant strains and their respective parental strains. Also in the competition experiments, the addition of FbsA protein or mAb 5H2 did not alter the invasion index. This indicates, that FbsA-mediated adherence of S. agalactiae is a prerequisite for subsequent host cell entry, which itself is independent of FbsA. This hypothesis is supported by our observation that FbsA-coated latex beads bound in high number to epithelial cells but were only rarely seen in the process of internalization by host cells. Furthermore, plasmid-mediated fbsA expression did not allow L. lactis to enter epithelial cells. Thus, our findings suggest that FbsA is not sufficient to promote the invasion of S. agalactiae into epithelial cells. Interestingly, the fibrinogen-binding protein FbsB was recently shown to mediate the invasion of S. agalactiae into epithelial cells (19). Thus, fibrinogen-binding proteins appear to play in S. agalactiae a prominent role in both host cell adherence and invasion. The FbsB protein, however, is not the only invasin in S. agalactiae. Also the C5a peptidase (7), the hemolysin CylE (11), and protein Spb1, being unique to serotype III-3 (1), have been shown to play a role in the entry of S. agalactiae into host cells. This indicates, that after FbsA-mediated adherence, different proteins can promote the entry of S. agalactiae into host cells.

Although the present and previous studies convincingly demonstrate the binding of FbsA to human fibrinogen (44), the host molecules that allow FbsA-mediated adherence remain to be determined. Externally added fibrinogen significantly inhibited the adherence of S. agalactiae to epithelial cells, however, it also caused a dose-dependent clumping of the bacteria (19). Thus, the inhibition of streptococcal adherence may be caused by the clumping of the bacteria. Host cell adherence was also unaffected by the addition of anti-fibrinogen antibodies. However, these antibodies neither blocked the binding of the bacteria to fibrinogen, suggesting the binding of FbsA to a region within human fibrinogen, which is too conserved in different species to allow the production of antibodies. Interestingly, mAb 5H2, directed against the fibrinogen-binding repeat region of FbsA competitively blocked the adherence of S. agalactiae to epithelial cells. This demonstrates that the repeat region of FbsA is involved in the streptococcal host cell adherence. Of note, mAb 2B1, which binds to the repeat region of FbsA without interfering with its fibrinogen-binding, did not block the adherence of S. agalactiae to epithelial cells. This result indicates, that fibrinogen-binding domains in the repeat region of FbsA are also involved in host cell adherence. Thus, binding of FbsA to fibrinogen on the surface of human cells might play a role in the colonization of epithelial surfaces. Numerous studies have demonstrated the synthesis of fibrinogen by the epithelial cell line A549, used in the present study (16, 17). However, only 10-20% of the secreted fibrinogen is directed to the apical side of A549 cells (16). Therefore, only a small amount of fibrinogen would be available for FbsA-mediated adherence. However, the pathogenic protozoan Pneumocystis carinli was shown to adhere to apically-located fibrinogen of A549 cells (46), indicating sufficient amounts of this protein on the apical side of lung cells to allow the binding of pathogenic organisms. Besides fibrinogen-mediated host cell adherence, the FbsA protein may alternatively bind to a different ligand on the surface of epithelial cells. Interestingly, the fibrinogen-binding protein CIfB from S. aureus was recently shown to interact with cytokeratin 10 on the surface of eukaryotic cells (36). Also the fibrinogen-binding protein CIfA from S. aureus was found to interact with a platelet membrane protein that is distinct from fibrinogen (45). These findings demonstrate that bacterial fibrinogen-binding proteins may interact with distinct ligands on the host cell surface.

The following references referred to above are incorporated herein by reference as if set forth in the present application in their entirety:
1. Adderson, E. E., S. Takahashi, Y. Wang, J. Armstrong, D. V. Miller, and J. F. Bohnsack. 2003. Subtractive hybridization identifies a novel predicted protein mediating epithelial cell invasion by virulent serotype III group B Streptococcus agalactiae. Infect.Immun. 71:6857-6863.
2. Bagg, J., I. R. Poxton, D. M. Weir, and P. W. Ross. 1982. Binding of type-III group-B streptococci to buccal epithelial cells. J.Med.Microbiol. 15:363-72.
3. Baker, C. J. and M. S. Edwards. 1995. Group B streptococcal infections, p. 980-1054. In J. S. a. J. O. K. Remington (ed.), Infectious diseases of the fetus and newborn infant. W. B. Saunders, Philadelphia.
4. Beckmann, C., J. D. Waggoner, T. O. Harris, G. S. Tamura, and C. E. Rubens. 2002. Identification of novel adhesins from Group B streptococci by use of phage display reveals that C5a peptidase mediates fibronectin binding. Infect.Immun. 70:2869-2876.
5. Broughton, R. A. and C. J. Baker. 1983. Role of adherence in the pathogenesis of neonatal group B streptococcal infection. Infect.Immun. 39:837-843.
6. Bulgakova, T. N., K. B. Grabovskaya, M. Ryc, and J. Jelinkova. 1986. The adhesin structures involved in the adherence of group B streptococci to human vaginal cells. Folia Microbiol.(Praha) 31:394-401.
7. Cheng, Q., D. Stafslien, S. S. Purushothaman, and P. Cleary. 2002. The group B streptococcal C5a peptidase is both a specific protease and an invasin.

Infect.Immun. 70:2408-2413.
8. Chhatwal, G. S., C. Lammier, and H. Blobel. 1984. Guanidine extraction enhances the binding of human fibrinogen to group- B streptococci. Med.Microbiol.Immunol.(Berl) 173:19-27.
9. Courtney, H. S., Y. Li, J. B. Dale, and D. L. Hasty. 1994. Cloning, sequencing, and expression of a fibronectin/fibrinogen-binding protein from group A streptococci. Infect.Immun. 62:3937-3946.
10. Courtney, H. S., S. Liu, J. B. Dale, and D. L. Hasty. 1997. Conversion of M serotype 24 of Streptococcus pyogenes to M serotypes 5 and 18: effect on resistance to phagocytosis and adhesion to host cells. Infect.Immun. 65:2472-2474.
11. Doran, K. S., J. C. Chang, V. M. Benoit, L. Eckmann, and V. Nizet. 2002. Group B streptococcal beta-hemolysin/cytolysin promotes invasion of human lung epithelial cells and the release of interleukin-8. J.Infect.Dis. 185:196-203.
12. Dubendorff, J. W. and F. W. Studier. 1991. Controlling basal expression in an inducible T7 expression system by blocking the target T7 promoter with lac repressor. J.Mol.Biol. 219:45-59.
13. Elsinghorst, E. A. 1994. Measurement of invasion by gentamycin resistance, p. 405-419. In J. N. Abelson and M. I. Simon (eds.), Bacterial Pathogenesis Part B. Academic Press.
14. Gasson, M. J. 1983. Plasmid complements of Streptococcus lactis NCDO 712 and other lactic streptococci after protoplast-induced curing. J.Bacteriol. 154:1-9.
15. Goldschmidt, J. C., Jr. and C. Panos. 1984. Teichoic acids of Streptococcus agalactiae: chemistry, cytotoxicity, and effect on bacterial adherence to human cells in tissue culture. Infect.Immun. 43:670-677.
16. Guadiz, G., L. A. Sporn, R. A. Goss, S. O. Lawrence, V. J. Marder, and P. J. Simpson-Haidaris. 1997. Polarized secretion of fibrinogen by lung epithelial cells. Am.J.Respir.Cell Mol.Biol. 17:60-69.
17. Guadiz, G., L. A. Sporn, and P. J. Simpson-Haidaris. 1997. Thrombin cleavage-independent deposition of fibrinogen in extracellular matrices. Blood 90:2644-2653.
18. Gutekunst, H., B. E. Eikmanns, and D. J. Reinscheid. 2003. Analysis of RogB-controlled virulence mechanisms and gene expression in Streptococcus agalactiae. Infect.Immun 71:5056-5064.
19. Gutekunst, H., B. E. Eikmanns, and D. J. Reinscheid. 2004. The novel fibrinogen-binding protein FbsB promotes the invasion of Streptococcus agalactiae into epithelial cells. Infect.Immun.
20. Hanahan, D. 1985. Studies on transformation of Escherichia coli with plasmids.

J.Mol.Biol. 166:557-580.
21. Hay, E. D. 1991. Cell biology of extracellular matrix. Plenum Press, New York.
22. Hunter, W. H. and F. C. Greenwood. 1962. Preparation of iodine-131 labelled human growth hormone of high specific activity. Nature 194:495-496.
23. Husmann, L. K., J. R. Scott, G. Lindahl, and L. Stenberg. 1995. Expression of the Arp protein, a member of the M protein family, is not sufficient to inhibit phagocytosis of Streptococcus pyogenes. Infect.Immun. 63:345-348.
24. Katerov, V., A. Andreev, C. Schalen, and A. A. Totolian. 1998. Protein F, a fibronectin-binding protein of Streptococcus pyogenes, also binds human fibrinogen: isolation of the protein and mapping of the binding region. Microbiology 144 (Pt 1): 119-126.
25. Kotarsky, H., A. Thern, G. Lindahl, and U. Sjobring. 2000. Strain-specific restriction of the antiphagocytic property of group A streptococcal M proteins. Infect.Immun. 68:107-112.
26. Lammler, C., G. S. Chhatwal, and H. Blobel. 1983. Binding of human fibrinogen and its polypeptide chains to group B streptococci. Med.Microbiol.Immunol.(Berl) 172:149-153.
27. Maguin, E., H. Prevost, S. Ehrlich, and A. Gruss. 1996. Efficient insertional mutagenesis in lactococci and other gram-positive bacteria. J.Bacteriol. 178:931-935.
28. Mardh, P. A. and L. Westtom. 1976. Adherence of bacterial to vaginal epithelial cells. Infect.Immun. 13:661-666.
29. Massey, R. C., M. N. Kantzanou, T. Fowler, N. P. Day, K. Schofield, E. R. Wann, A. R. Berendt, M. Hook, and S. J. Peacock. 2001. Fibronectin-binding protein A of Staphylococcus aureus has multiple, substituting, binding regions that mediate adherence to fibronectin and invasion of endothelial cells. Cell Microbiol. 3:839-851.
30. McDevitt, D., P. Francois, P. Vaudaux, and T. J. Foster. 1994. Molecular characterization of the clumping factor (fibrinogen receptor) of Staphylococcus aureus. Mol.Microbiol. 11:237-248.
31. Miyazaki, S., O. Leon, and C. Panos. 1988. Adherence of Streptococcus agalactiae to synchronously growing human cell monolayers without lipoteichoic acid involvement. Infect.Immun. 56:505-512.
32. Nealon, T. J. and S. J. Mattingly. 1984. Role of cellular lipoteichoic acids in mediating adherence of serotype III strains of group B streptococci to human embryonic, fetal, and adult epithelial cells. Infect.Immun. 43:523-530.
33. Nealon, T. J. and S. J. Mattingly. 1985. Kinetic and chemical analyses of the biologic significance of lipoteichoic acids in mediating adherence of serotype III group B streptococci. Infect.Immun. 50:107-115.
34. Nguyen, M. D. and P. J. Simpson-Haidaris. 2000. Cell type-specific regulation of fibrinogen expression in lung epithelial cells by dexamethasone and interleukin-1beta. Am.J.Respir.Cell Mol.Biol. 22:209-217.
35. Ni, E. D., S. Perkins, P. Francois, P. Vaudaux, M. Hook, and T. J. Foster. 1998. Clumping factor B (CIfB), a new surface-located fibrinogen-binding adhesin of Staphylococcus aureus. Mol.Microbiol. 30:245-257.
36. O'Brien, L. M., E. J. Walsh, R. C. Massey, S. J. Peacock, and T. J. Foster. 2002. Staphylococcus aureus clumping factor B (CIfB) promotes adherence to human type I cytokeratin 10: implications for nasal colonization. Cell Microbiol. 4:759-770.
37. Palma, M., S. Nozohoor, T. Schennings, A. Heimdahl, and J. I. Flock. 1996. Lack of the extracellular 19-kilodalton fibrinogen-binding protein from Staphylococcus aureus decreases virulence in experimental wound infection. Infect.Immun. 64:5284-5289.
38. Perez-Casal, J., M. G. Caparon, and J. R. Scott. 1992. Introduction of the emm6 gene into an emm-deleted strain of Streptococcus pyogenes restores its ability to resist phagocytosis. Res.Microbiol. 143:549-558.
39. Podbielski, A., N. Schnitzler, P. Beyhs, and M. D. Boyle. 1996. M-related protein (Mrp) contributes to group A streptococcal resistance to phagocytosis by human granulocytes. Mol.Microbiol. 19:429-441.
40. Que, Y. A., J. A. Haefliger, P. Francioli, and P. Moreillon. 2000. Expression of Staphylococcus aureus clumping factor A in Lactococcus lactis subsp. cremoris using a new shuttle vector. Infect.Immun. 68:3516-3522.
41. Reinscheid, D. J., C. Stoesser, K. Moeller, K. Ehlert, R. W. Jack, B. E. Eikmanns, and G. S. Chhatwal. 2002. Influence of proteins Bsp and FemH on cell shape and peptidoglycan composition in group B streptococcus. Microbiol. 148:3245-3254.
42. Rubens, C. E., H. V. Raff, J. C. Jackson, E. Y. Chi, J. T. Bielitzki, and S. L. Hillier. 1991. Pathophysiology and histopathology of group B streptococcal sepsis in Macaca nemestrina primates induced after intraamniotic inoculation: evidence for bacterial cellular invasion. J.Infect.Dis. 164:320-330.
43. Samen, U., B. Gottschalk, B. J. Eikmanns, and D. J. Reinscheid. 2004. Relevance of Peptide Uptake Systems to the Physiology and Virulence of Streptococcus agalactiae. J.Bacteriol. 186:1398-1408.
44. Schubert, A., K. Zakikhany, M. Schreiner, R. Frank, B. Spellerberg, B. E. Eikmanns, and D. J. Reinscheid. 2002. A fibrinogen receptor from group B streptococcus interacts with fibrinogen by repetitive units with novel ligand binding sites. Mol.Microbiol. 46:557-569.
45. Siboo, I. R., A. L. Cheung, A. S. Bayer, and P. M. Sullam. 2001. Clumping factor A mediates binding of Staphylococcus aureus to human platelets. Infect.Immun. 69:3120-3127.
46. Simpson-Haidaris, P. J., M. A. Courtney, T. W. Wright, R. Goss, A. Harmsen, and F. Gigliotti. 1998. Induction of fibrinogen expression in the lung epithelium during Pneumocystis carinii pneumonia. Infect.Immun. 66:4431-4439.
47. Spellerberg, B. 2000. Pathogenesis of neonatal Streptococcus agalactiae infections. Microbes.Infect. 2:1733-1742.
48. Spellerberg, B., E. Rozdzinski, S. Martin, J. Weber-Heynemann, N. Schnitzler, R. Lutticken, and A. Podbielski. 1999. Lmb, a protein with similarities to the LraI adhesin family, mediates attachment of Streptococcus agalactiae to human laminin. Infect.Immun. 67:871-878.
49. Tamura, G. S., J. M. Kuypers, S. Smith, H. Raff, and C. E. Rubens. 1994. Adherence of group B streptococci to cultured epithelial cells: roles of environmental factors and bacterial surface components. Infect.Immun. 62:2450-2458.
50. Tamura, G. S. and A. Nittayajarn. 2000. Group B streptococci and other gram-positive cocci bind to cytokeratin 8. Infect.Immun. 68:2129-2134.
51. Tamura, G. S. and C. E. Rubens. 1995. Group B streptococci adhere to a variant of fibronectin attached to a solid phase. Mol.Microbiol. 15:581-589.
52. Taschner, S., A. Meinke, A. von Gabain, and A. P. Boyd. 2002. Selection of peptide entry motifs by bacterial surface display. Biochem.J. 367:393-402.
53. Teti, G., F. Tomasello, M. S. Chiofalo, G. Orefici, and P. Mastroeni. 1987. Adherence of group B streptococci to adult and neonatal epithelial cells mediated by lipoteichoic acid. Infect.Immun. 55:3057-3064.
54. Thern, A., M. Wastfelt, and G. Lindahl. 1998. Expression of two different antiphagocytic M proteins by Streptococcus pyogenes of the OF+ lineage. J.Immunol. 160:860-869.
55. Waite, D. C., E. J. Alper, and B. J. Mady. 1996. Adult group B streptococcal disease. Ann.Intern.Med. 125:152-153.
56. Wann, E. R., S. Gurusiddappa, and M. Hook. 2000. The fibronectin-binding MSCRAMM FnbpA of Staphylococcus aureus is a bifunctional protein that also binds to fibrinogen. J.Biol.Chem. 275:13863-13871.
57. Wells, J. M., P. W. Wilson, and R. W. Le Page. 1993. Improved cloning vectors and transformation procedure for Lactococcus lactis. J.Appl.Bacteriol. 74:629-636.
58. Whitnack, E. and E. H. Beachey. 1982. Antiopsonic activity of fibrinogen bound to M protein on the surface of group A streptococci. J.Clin.Invest 69:1042-1045.
59. Wibawan, I. T., C. Lammler, and F. H. Pasaribu. 1992. Role of hydrophobic surface proteins in mediating adherence of group B streptococci to epithelial cells. J.Gen.Microbiol. 138 ( Pt 6):1237-1242.
60. Winram, S. B., M. Jonas, E. Chi, and C. E. Rubens. 1998. Characterization of group B streptococcal invasion of human chorion and amnion epithelial cells In vitro. Infect.Immun. 66:4932-4941.
61. Zawaneh, S. M., E. M. Ayoub, H. Baer, A. C. Cruz, and W. N. Spellacy. 1979. Factors influencing adherence of group B streptococci to human vaginal epithelial cells. Infect.Immun. 26:441-447.

It will be appreciated by those of skill in the art that the compositions and methods as described above are only exemplary of the present invention, and that those of ordinary skill in the art should, in light of the present disclosure, appreciate that many changes can be made in the specific embodiments which are disclosed and still obtain a like or similar result without departing from the spirit and scope of the invention.

1. An isolated antibody which binds to the FbsA protein from S. agalactiae.

2. The antibody of claim 1 wherein said antibody is a monoclonal antibody.

3. The antibody of claim 1 wherein said antibody is a polyclonal antibody.

4. The antibody of claim 1 wherein the antibody is raised against the fibrinogen binding domain from S. agalactiae.

5. The antibody of claim 1 wherein the antibody is raised against the repeat region of the fibrinogen binding domain from S. agalactiae.

6. The antibody of claim 1 wherein the antibody is able to prevent adherence of S. agalactiae to a human or animal host cell.

7. The antibody of claim 1 wherein the antibody is able to prevent adherence of S. agalactiae to fibrinogen.

8. The antibody of claim 1 wherein the antibody is able to treat or prevent S. agalactiae infection.

9. The antibody of claim 1, wherein said antibody is suitable for parenteral, oral, intranasal, subcutaneous, aerosolized or intravenous administration in a human or animal.

10. The antibody of claim 2 wherein the monoclonal antibody is of a type selected from the group consisting of murine, chimeric, humanized and human monoclonal antibodies.

11. The antibody of claim 2 wherein the antibody is a single chain monoclonal antibody.

12. The antibody of claim 1 wherein said antibody is raised against the FbsA protein of S. agalactiae.

13. Isolated antisera containing an antibody according to claim 1.

14. A pharmaceutical composition comprising the antibody of claim 1 in an amount effective to prevent adherence of S. agalactiae to host cells, and a pharmaceutically acceptable vehicle, carrier or excipient.

15. A diagnostic kit comprising an antibody according to claim 1 and means for detecting binding by that antibody.

16. A diagnostic kit according to claim 15 wherein said means for detecting binding comprises a detectable label that is linked to said antibody.

17. A method of diagnosing an infection of S. agalactiae comprising adding an antibody according to claim 1 to a sample suspected of being infected with S. agalactiae, and determining if antibodies have bound to the sample.

18. A method of treating or preventing an infection of S. agalactiae comprising administering to a human or animal patient an effective amount of the antibody according to claim 1.

19. A method of inducing an immunological response comprising administering to a human or animal an immunogenic amount of the FbsA protein of S. agalactiae.

20. A method of treating or preventing platelet aggregation comprising administering to a human or animal patient an effective amount of the antibody according to claim 1.

21. An isolated antibody which binds to the fibrinogen binding region of the FbsA protein of S. agalactiae.

22. The antibody of claim 21 wherein said antibody is a monoclonal antibody.

23. The antibody of claim 21 wherein said antibody is a polyclonal antibody.

24. The antibody of claim 21 wherein said antibody is able to prevent the adherence of S. agalactiae to a host cell.

25. The antibody of claim 21 wherein said antibody is able to prevent the adherence of S. agalactiae to fibrinogen.

26. The antibody of claim 21 wherein said antibody is able to prevent the adherence of S. agalactiae to an indwelling medical device or implant.

27. The antibody of claim 21 wherein said antibody is able to bind to the repeat region of the fibrinogen binding domain of FbsA.

28. Isolated antisera containing an antibody according to claim 21.

29. A pharmaceutical composition comprising the antibody of claim 21 and a pharmaceutically acceptable vehicle, carrier or excipient.

30. A diagnostic kit comprising an antibody according to claim 21 and means for detecting binding by that antibody.

31. A vaccine comprising the FbsA protein from S. agalactiae in an amount effective to elicit antibodies against the FbsA protein, and a pharmaceutically acceptable vehicle, carrier or excipient.

32. The vaccine of claim 31 wherein said vaccine is capable of generating antibodies which block the adherence of S. agalactiae to host cells.

33. A vaccine comprising the fibrinogen binding region of the FbsA protein from S. agalactiae in an amount effective to elicit antibodies against the FbsA protein, and a pharmaceutically acceptable vehicle, carrier or excipient.

34. The vaccine of claim 33 wherein said vaccine is capable of generating antibodies which block the adherence of S. agalactiae to host cells.

35. An isolated antibody which binds to the repeat region of the FbsA protein of S. agalactiae.

36. The antibody of claim 35 wherein said antibody is able to prevent the adherence of S. agalactiae to a host cell.

37. The antibody of claim 35 wherein said antibody is able to prevent the adherence of S. agalactiae to fibrinogen.

38. The antibody of claim 35 wherein said antibody is a monoclonal antibody.

39. The antibody of claim 35 wherein said antibody is a polyclonal antibody.

40. A pharmaceutical composition comprising the antibody of claim 35 and a pharmaceutically acceptable vehicle, carrier or excipient.


SEQUENCE LISTING (2 sequences)

SEQ ID NO: 1 - DNA, 38 bp, Streptococcus agalactiae
gtttagtggatccgaagtaaggagaaaattaattgttc

SEQ ID NO: 2 - DNA, 18 bp, Streptococcus agalactiae
atcccatataatgacctc